SEQUENCE LISTING (1 sequence)

SEQ ID NO: 1 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

(19) United States
(12) Patent Application Publication
(10) Pub. No.: US 2026/0132396 A1
(43) Pub. Date: May 14, 2026

(54) PERFORMANCE-ENHANCED PROTEASE VARIANTS

(71) Applicant: Henkel AG & Co. KGaA, Dusseldorf (DE)

(72) Inventors: Christian Degering, Erkrath (DE); Susanne Wieland, Zons/Dormagen (DE); Inken Prueser, Dusseldorf (DE); Vedat Durmaz, Saarbrücken (DE); Christian Gruber, Weiz (AT); Georg Steinkellner, Kainach bei Voitsberg (AT)

(21) Appl. No.: 19/384,576

(22) Filed: Nov. 10, 2025

(30) Foreign Application Priority Data
Nov. 14, 2024 (DE) 102024133317.0

(51) Int. Cl. C12N 9/54; C11D 3/386

(52) U.S. Cl. CPC C12N 9/54; C11D 3/386

(57) ABSTRACT

The invention relates to proteases that exhibit proteolytic activity and comprise an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, and 98% identical to the amino acid sequence indicated in SEQ ID NO:1 over its entire length, wherein the protease comprises, based on the numbering according to SEQ ID NO:1, at least one amino acid substitution selected from the group consisting of N97E, R99E, R99I, R99N, R99V, R99T, D127E and M211C in at least one of the positions that correspond to positions 97.3 99, 127 and 211. Such proteases are suitable for use in washing and cleaning agents, in particular liquid textile washing agents, and have improved cleaning performance in comparison with a reference protease. The invention also relates to the use of said proteases and methods in which they are used, and to washing and cleaning agents containing said proteases, in particular liquid textile washing agents.

### CROSS REFERENCE TO RELATED APPLICATIONS

This application claims the benefit of German Patent Publication No. 102024133317.0, filed Nov. 14, 2024, which is incorporated by reference in its entirety herein.

### SEQUENCE LISTING

The Instant Application contains a Sequence Listing which has been submitted electronically in XML format and is hereby incorporated by reference in its entirety. Said XML copy, created on Sep. 5, 2025, is named “HN10056US” and is 2,235 bytes in size.

### FIELD OF THE INVENTION

The invention is in the field of enzyme technology. The invention relates to proteases, the amino acid sequence of which has been altered in particular when using them in washing and cleaning agents, in particular liquid textile washing agents, in order to improve their cleaning performance, and to the nucleic acids coding therefor, and to the production thereof. The invention further relates to the use of said proteases and methods in which they are used, and to washing and cleaning agents containing said proteases, in particular liquid washing and cleaning agents, particularly preferably liquid textile washing agents.

Proteases are among the technically most important enzymes. For washing and cleaning agents, they are the longest established enzymes and are contained in virtually all modern, high-performance washing and cleaning agents. They cause the degradation of protein-containing stains on the articles to be cleaned. In turn, proteases of the subtilisin type (subtilases, subtilopeptidases, EC 3.4.21.62) are particularly important, which proteases are serine proteases due to the catalytically active amino acids. They act as unspecific endopeptidases and hydrolyze any acid amide bonds within peptides or proteins. Their optimum pH is usually in the distinctly alkaline range. The article “Subtilases: Subtilisin-like Proteases” by R. Siezen, pages 75-95 in “Subtilisin enzymes,” published by R. Bott and C. Betzel, New York, 1996, for example, gives an overview of this family. Subtilases are naturally formed by microorganisms. In particular, the subtilisins formed and secreted by the Bacillus species are the most significant group of subtilases.

Examples of subtilisin-type proteases that are preferably used in washing and cleaning agents are the subtilisins BPN′ and Carlsberg, protease PB92, subtilisins 147 and 309, the alkaline protease from Bacillus lentus, in particular Bacillus lentus DSM 5483, subtilisin DY and the enzymes thermitase, proteinase K and proteases TW3 and TW7, which are to be classified as subtilases, but no longer as subtilisins in the narrower sense, as well as variants of said proteases that have an amino acid sequence which is altered compared to the starting protease. Proteases are altered in a targeted or random manner by methods known from the prior art and are thus optimized for use in washing and cleaning agents, for example. This includes point, deletion or insertion mutagenesis, or fusion with other proteins or protein parts. Thus, appropriately optimized variants are known for most proteases known from the prior art. WO 2017/215925, for example, discloses a protease from Bacillus gibsonii or variants thereof, which is intended for washing and cleaning agents, in particular dishwasher detergents.

In general, only selected proteases are suitable for use in liquid, surfactant-containing preparations in any case. Many proteases do not exhibit sufficient catalytic performance in such preparations or they are not sufficiently stable. For the use of proteases in washing and cleaning agents, therefore, a high catalytic activity and stability under conditions such as are present during a washing process is particularly desirable. Consequently, protease- and surfactant-containing liquid formulations from the prior art are disadvantageous in that the proteases present, under standard washing conditions, do not exhibit satisfactory proteolytic activity and/or are not sufficiently storage-stable, and the formulations therefore do not exhibit optimal cleaning performance on protease-sensitive stains. Protease-sensitive stains are preferably selected from the group consisting of stains containing blood, egg (yolk), milk, meat and other proteins.

The most important criterion in cleaning textiles and/or hard surfaces is the cleaning performance on a wide variety of stains. Even if the cleaning performance of the washing and cleaning agents used today is generally good, the general trend of increasingly using low-temperature programs, however, poses the problem that many of the conventional washing and cleaning agents have insufficient cleaning performance for stubborn stains. In particular, egg (yolk)-containing stains pose a challenge. Such an inadequate cleaning performance leads to dissatisfaction among consumers and to the fact that such stains are pretreated by consumers, which in turn increases the consumption of water and energy. Stubborn stains are also usually dealt with in typical washing and cleaning agents by means of further active cleaning substances in the washing and cleaning agent, such as phosphonates and/or phosphates. Sustainability aspirations and concerns regarding the environmental friendliness of phosphates and phosphonates in washing and cleaning agents mean that increasingly more washing and cleaning agents are being developed which contain little (or less) or no phosphate-containing and/or phosphonate-containing compounds.

There is still a need to improve the cleaning performance of enzyme-containing, in particular protease-containing, washing and cleaning agents, in particular liquid textile washing agents, in particular with regard to the cleaning performance on protease-sensitive stains. The present invention pays particular attention to improving the cleaning performance of protease-containing washing and cleaning agents, in particular liquid textile washing agents, on stains containing protein, in particular containing blood, egg (yolk), milk and/or meat, preferably containing egg (yolk) and/or milk. The improvement in cleaning performance is preferably within a temperature range from approximately 20° C. to approximately 60° C., preferably from approximately 20° C. to approximately 40° C., more preferably from about 20° C. to approximately 30° C., particularly preferably approximately 20° C. Furthermore, there is still a need to improve the storage stability of the protease in a protease-containing washing and cleaning agent, in particular a liquid textile washing agent.

Surprisingly, it has now been discovered that a protease from Bacillus gibsonii or a protease that is sufficiently similar thereto (in terms of sequence identity) that, based on the numbering according to SEQ ID NO:1, has at least one amino acid substitution selected from the group consisting of N97E, R99E, R99I, R99N, R99V, R99T, D127E and M211C in at least one of the positions corresponding to positions 97, 99, 127 and 211, is improved in terms of its cleaning performance in comparison with a reference protease (in particular the wild-type protease according to SEQ ID NO:1), and is therefore particularly suitable for use in washing and cleaning agents, in particular in liquid textile washing agents.

The invention therefore relates to a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease comprises, based on the numbering according to SEQ ID NO:1, at least one amino acid substitution selected from the group consisting of N97E, R99E, R99I, R99N, R99V, R99T, D127E and M211C in at least one of the positions that correspond to positions 97, 99, 127 and 211.

The invention preferably relates to a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease comprises, based on the numbering according to SEQ ID NO:1, at least two amino acid substitutions selected from the group consisting of N97E, R99E, R99I, R99N, R99V, R99T, D127E and M211C in at least two of the positions that correspond to positions 97, 99, 127 and 211.

The invention preferably relates to a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease comprises, based on the numbering according to SEQ ID NO:1, at least three amino acid substitutions selected from the group consisting of N97E, R99E, R99I, R99N, R99V, R99T, D127E and M211C in at least three of the positions that correspond to positions 97, 99, 127 and 211.

The invention preferably relates to a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease comprises, based on the numbering according to SEQ ID NO:1, at least four amino acid substitutions selected from the group consisting of N97E, R99E, R99I, R99N, R99V, R99T, D127E and M211C in at least four of the positions that correspond to positions 97, 99, 127 and 211.

The invention further relates to a method for producing a protease, involving introducing at least one amino acid substitution selected from the group consisting of N97E, R99E, R99I, R99N, R99V, R99T, D127E and M211C into a starting molecule that has an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% or 100% identical to the amino acid sequence indicated in SEQ ID NO:1 over its entire length in at least one of the positions which, based on the numbering according to SEQ ID NO:1, correspond to positions 97, 99, 127 and 211.

A protease within the meaning of the present patent application therefore comprises both the protease as such and a protease produced using a method according to the invention. All statements relating to the protease therefore relate both to the protease as such, as well as to the proteases produced by means of corresponding methods, and to the corresponding methods, in particular production methods of the protease.

Further aspects of the invention relate to the nucleic acids encoding these proteases, to non-human host cells containing proteases or nucleic acids according to the invention, and washing and cleaning agents comprising proteases according to the invention, preferably liquid washing and cleaning agents, particularly preferably liquid textile washing agents, to washing and cleaning methods, and to the use of proteases according to the invention in washing and/or cleaning agents for removing protease-sensitive stains, in particular stains containing egg (yolk), milk, blood and/or meat, preferably stains containing egg (yolk) and/or milk.

These and other aspects, features and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

Unless indicated otherwise, all percentages are indicated in terms of weight percent (wt. %).

Numerical ranges that are indicated in the format “from x to y” also include the stated values. If several preferred numerical ranges are specified in this format, it is readily understood that any ranges resulting from the combination of the various endpoints are also included.

“At least one,” as used herein, means one or more, i.e., 1, 2, 3, 4, 5, 6, 7, 8, 9, 10, 11, 12, 13, 14 or more.

The term “washing and cleaning agent” or “washing or cleaning agent,” as used herein, is synonymous with the term “agent” and denotes a composition for cleaning textiles and/or hard surfaces, in particular dishes, as explained in the description.

“Approximately,” “about,” or “roughly,” as used herein in reference to a numerical value, refers to the corresponding numerical value ±10%, preferably ±5%.

“Substantially free from” means that the composition or the agent contains less than 2 wt. %, preferably less than 1 wt. %, more preferably less than 0.5 wt. %, and particularly preferably less than 0.1 wt. %, of the corresponding substance, based on the total weight of the composition/agent.

“Liquid,” as used herein, includes liquids and gels as well as pasty compositions. It is preferred that the liquid compositions be flowable and pourable at room temperature, but it is also possible for them to have a limit of liquidity.

According to the definition of the invention, a substance, e.g., a composition or an agent, is solid if it is solid at 25° C. and 1013 mbar.

According to the definition of the invention, a substance, e.g., a composition or an agent, is liquid if it is liquid at 25° C. and 1013 mbar. Liquid also includes gel form.

“Variant,” as used herein, refers to naturally or artificially generated variations of a native protease which has an amino acid sequence which is modified from the reference form.

The term “textile” as used herein means any textile material, including yarns, yarn precursors, fibers, nonwovens, natural materials, synthetic materials and all other textile materials, fabrics made from these materials, and products made from fabrics (e.g., garments and other articles). The textile or fabric can be in the form of knits, wovens, denims, nonwovens, felts, yarns and terry cloth. The textile can be based on cellulose, for example natural cellulose fibers such as cotton, flax/linen, jute, ramie, sisal or coconut fibers, or synthetically produced cellulose fibers (e.g., from pulp) such as viscose/rayon, cellulose acetate fibers (tricell), lyocell or mixtures thereof. The textile or fabric can also consist of non-cellulosic fibers, e.g., natural polyamides such as wool, camel, cashmere, mohair, rabbit hair and silk, or synthetic polymers such as nylon, aramid, polyester, acrylic, polypropylene and spandex/elastane or mixtures thereof, as well as mixtures of cellulosic fibers and non-cellulosic fibers. Examples of blends are blends of cotton and/or rayon/viscose having one or more accompanying materials such as wool, synthetic fibers (e.g., polyamide fibers, acrylic fibers, polyester fibers, polyvinyl chloride fibers, polyurethane fibers, polyurea fibers, aramid fibers) and/or cellulose-containing fibers (e.g., rayon/viscose, ramie, flax/linen, jute, cellulose acetate fibers, Lyocell). The fabric can be conventional washable laundry, e.g., soiled household laundry. When the term “fabric” or “garment” is used, it is intended to include the broader term “textiles.”

The present invention is based on the inventors' surprising finding that amino acid substitutions at the positions described herein result in improved cleaning performance of this modified protease in washing and cleaning agents, in particular liquid textile washing agents, preferably at low temperatures of from approximately 20° C. to approximately 40° C., preferably approximately 20° C.

In preferred embodiments, the modifications according to the invention in at least one of the positions corresponding to positions 97, 99, 127 and 211, at least one amino acid substitution selected from the group consisting of N97E, R99E, R99I, R99N, R99V, R99T, D127E and M211C leads to an improved cleaning performance of this altered protease in washing and cleaning agents, in particular textile washing agents, on at least one protease-sensitive stain under standard washing conditions.

In preferred embodiments, the modifications according to the invention in at least one of the positions corresponding to positions 97, 99, 127 and 211, at least one amino acid substitution selected from the group consisting of N97E, R99E, R99I, R99N, R99V, R99T, D127E and M211C leads to an improved cleaning performance of this modified protease in washing and cleaning agents, in particular liquid textile washing agents, on stains containing egg (yolk), milk, blood and/or meat, in particular egg (yolk) and/or milk, under standard washing conditions.

This is surprising in particular in that no protease with such modifications has been described thus far for use in washing or cleaning agents, in particular liquid textile washing agents. In particular, such proteases modified according to the invention were not described in connection with improved cleaning performance on stains containing egg (yolk), milk, blood and/or meat, in particular egg (yolk) and/or milk.

In preferred embodiments, the protease according to the invention has at least one amino acid substitution selected from the group consisting of N97E, R99E, R99I, R99N, R99V, R99T, D127E and M211C in at least one of the positions that correspond to positions 97, 99, 127 and 211, wherein the at least one amino acid substitution leads to an improved cleaning performance of this modified protease in washing and cleaning agents, in particular liquid textile washing agents, on at least one protease-sensitive stain.

In preferred embodiments, the protease according to the invention has at least one amino acid substitution selected from the group consisting of N97E, R99E, R99I, R99N, R99V, R99T, D127E and M211C in at least one of the positions that corresponds to positions 97, 99, 127 and 211, wherein the at least one amino acid substitution leads to an improved cleaning performance of this modified protease in washing and cleaning agents, in particular liquid textile washing agents, on stains containing egg (yolk), milk, blood and/or meat, in particular egg (yolk) and/or milk.

In preferred embodiments, the amino acid substitutions or amino acid substitution combinations according to the invention described herein lead to an improved cleaning performance of this modified protease in washing and cleaning agents, in particular liquid textile washing agents, on at least one protease-sensitive stain, preferably stains containing egg (yolk), milk, blood and/or meat, in particular egg (yolk) and/or milk. Proteases according to the invention consequently allow improved removal of at least one, preferably multiple, protease-sensitive stains on textiles and/or hard surfaces, in particular dishes. Typical protease-sensitive stains include stains containing egg (yolk), blood, milk, meat and other proteins, for example. Particularly preferably, proteases according to the invention enable improved removal of stains on textiles that contains egg (yolk), milk, blood and/or meat, in particular egg (yolk) and/or milk. An improvement in cleaning performance, in particular in proteolytic cleaning performance, in accordance with the invention is present when the protease exhibits improved cleaning performance compared with a reference protease (in particular the wild-type protease according to SEQ ID NO:1), as described herein, on at least one protease-sensitive stain, which is preferably selected from the group consisting of stains containing blood, egg (yolk), milk, meat and other proteins.

Within the context of the invention, washing or cleaning performance is understood to mean the ability of a washing or cleaning agent to partially or completely remove existing stains, i.e. the ability to lighten one or more stains, in particular on textiles, laundry or dishes. Within the scope of the invention, both the washing and/or cleaning agent comprising the protease, or the washing or cleaning liquor formed by said agent, and the protease itself have a particular cleaning performance. The cleaning performance of the enzyme thus contributes to the cleaning performance of the agent, or of the washing or cleaning liquor formed by the agent. The cleaning performance is preferably ascertained as described hereafter.

Washing or cleaning liquor is understood to mean the ready-to-use solution that contains the washing or cleaning agent and acts on textiles or fabric or hard surfaces, in particular dishes, and thus comes into contact with the stains present on textiles or fabrics or hard surfaces, in particular dishes. The washing or cleaning liquor is usually created when the washing or cleaning process begins and the washing or cleaning agent is diluted with water, e.g., in a dishwasher, a washing machine or in another suitable container.

The cleaning performance on textiles or fabrics can be determined in a washing system containing a washing agent in a dosage between 2.0 and 8.0 grams per liter of washing liquor. The proteases to be compared are used in the same concentration (based on active protein). The concentration of the protease in the washing agent intended for such a washing system is 0.001 to 0.1 wt. %, preferably 0.01 to 0.06 wt. %, relative to active protein.

A liquid reference washing agent for such a washing system may be composed, for example, as follows (all figures in wt. %): 4.4% alkyl benzene sulfonic acid, 5.6% further anionic surfactants, 2.4% C₁₂₋₁₈ Na salts of fatty acids (soaps), 4.4% non-ionic surfactants, 0.2% phosphonates, 1.4% citric acid, 0.95% NaOH, 0.01% defoamer, 2% glycerol, 0.08% preservatives, 1% ethanol, and the remainder being demineralized water. The dosage of the liquid washing agent is preferably between 3.0 and 6.0 grams per liter of washing liquor, e.g., 3.0, 3.2, 3.5, 3.7, 4.0, 4.5, 4.7, 4.9 or 5.9 grams per liter of washing liquor. The washing process preferably takes place in a pH range between pH 7 and pH 10.5, preferably between pH 8 and pH 9.

Alternatively, a liquid reference washing agent for such a washing system can be composed as follows (all information in wt. %): 0.3-0.5% xanthan gum, 0.2-0.4% anti-foaming agent, 6-7% glycerol, 0.3-0.5% ethanol, 4-7% FAEOS (fatty alcohol ether sulfate), 5-15% non-ionic surfactants, 5-15% anionic surfactants (LAS), 1% boric acid, 1-4% sodium citrate (dihydrate), 2-4% soda, 2-6% coconut fatty acids, 0.5-2.5% HEDP (1-hydroxyethane-(1,1-diphosphonic acid)), 0-0.4% PVP (polyvinylpyrrolidone), 0-0.15% optical brighteners, 0-0.001% dye, the remainder being demineralized water. The dosage of the liquid washing agent is preferably between 3.5 and 6.0 grams per liter of washing liquor, for example 4.7, 4.9 or 5.9 grams per liter of washing liquor. The washing process preferably takes place in a pH range between pH 8 and pH 10.5, preferably between pH 8 and pH 9.

A powdered reference washing agent for such a washing system can be composed as follows (all information in wt. %): 10% linear alkylbenzene sulfonate (sodium salt), 1.5% C₁₂₋₁₈ fatty alcohol sulfate (sodium salt), 2.0% C₁₂₋₁₈ fatty alcohol having 7 EO, 20% sodium carbonate, 6.5% sodium hydrogen carbonate, 4.0% amorphous sodium disilicate, 17% sodium carbonate peroxohydrate, 4.0% TAED, 3.0% polyacrylate, 1.0% carboxymethyl cellulose, 1.0% phosphonate, 27% sodium sulfate, remainder: foam inhibitors, optical brighteners, fragrances. The dosage of the powdered washing agent is preferably between 4.5 and 7.0 grams per liter of washing liquor, for example and particularly preferably 4.7 grams per liter of washing liquor, or 5.5, 5.9 or 6.7 grams per liter of washing liquor. Washing in a pH range between pH 9 and pH 11 is preferred.

The cleaning performance against stains on textiles (e.g. cotton or cotton blend fabric) is determined by measuring the degree of cleaning of the washed textiles. For example, the washing process can take place for 60 minutes at a temperature of approximately 20° C. or approximately 40° C. and the water can have a water hardness between 15.5° dH and 16.5°dH (German hardness). Within the scope of the invention, the cleaning performance is determined, for example, at 20° C. or 40° C. using a liquid washing agent as specified herein, wherein the washing process is preferably carried out for 60 minutes at 600 rpm.

The degree of whiteness, i.e., the lightening of the stains, as a measure of the cleaning performance is determined using optical measuring methods, preferably photometrically. A suitable device for this purpose is, for example, the Minolta CM508d spectrometer. Usually, the devices used for measurement are calibrated beforehand using a white standard, preferably a supplied white standard.

The cleaning performance of a dishwashing detergent can be determined in a system that contains an automatic dishwasher detergent in a dosage as specified herein as well as the protease according to the invention, wherein the proteases to be compared are used in the same concentration (based on active protein), and the cleaning performance with regard to tea, meat, spaghetti and/or creme brulee stains is determined according to the IKW method (Recommendations for the Quality Assessment of the Cleaning Performance of Dishwasher Detergents (Part B, Update 2015), sofwjournal, 142, 06/16, 34-48) in a Miele GSL (program 45° C., 21° dH). The concentration of the protease in the agent intended for this washing system is from 0.001 to 0.1 wt. %, preferably 0.01 to 0.06 wt. %, based on active, purified protein.

### Liquid Dishwashing Detergent (Two-Component Formulation)

| | Active substance |
|---|---|
| | content in wt. % |
| Enzyme phase (EP) - Preparation A | |
| Phosphonate (e.g., HEDP), if | 0.00-7.50 |
| permitted by regulations | |
| CaCl₂ | 0.05-1.50 |
| Amylase-containing enzyme composition (t.q.) | 0.00-4.00 |
| Protease-containing enzyme composition (t.q.) | 0.00001-10 |
| Sorbitol | 2.00-10.00 |
| Sulfonic acid group-containing polymer | 0.00-12.00 |
| Thickener (based on acrylate or xanthan gum) | 0.01-6.00 |
| GLDA or MGDA | 3.00-25.00 |
| KOH | 0.50-4.00 |
| Non-ionic surfactants | 1.00-6.00 |
| Sodium citrate | 2.00-20.00 |
| Zinc salt | 0.00-1.00 |
| Remainder (perfume, dyes, preservatives, | up to 100 |
| water, enzyme stabilizer) (wt. %) | |
| Alkali phase (AP) - Preparation B | |
| Phosphonate, if permitted by regulations | 0.00-7.50 |
| Thickener (acrylate or xanthan gum) | 0.01-6.00 |
| GLDA or MGDA | 3.00-25.00 |
| KOH | 0.50-4.00 |
| Soda | 5.00-20.00 |
| Monoethanolamine | 0.00-5.00 |
| Acrylate polymer | 0.00-3.00 |
| Sodium citrate | 2.00-20.00 |
| Remainder (perfume, dyes, preservatives, | up to 100 |
| water, etc.) (wt. %) | |

### Solid Dishwashing Detergent

| | Wt. % |
|---|---|
| Citrate, Na salt | 15-20 |
| Phosphonate (e.g., HEDP), if | 0-7.5 (2.5-7.5) |
| permitted by regulations | |
| MGDA, Na salt | 0-25 |
| Disilicate, Na salt | 5-35 |
| Soda | 10-25 |
| Silver protection | 0.0-1.0 |
| Percarbonate, Na salt | 10-15 |
| Bleach catalyst (preferably Mn-based) | 0.02-0.5 |
| Bleach activator (e.g., TAED) | 1-3 |
| Non-ionic surfactant(s), e.g., fatty | 2.5-10 |
| alcohol alkoxylate, preferably 20-40 | |
| EO, optionally end-capped | |
| Polycarboxylate | 4-10 |
| Cationic copolymer | 0-0.75 |
| Disintegrant - (e.g. crosslinked PVP) | 0-1.5 |
| Protease preparation (t.q.) | 0-5 |
| Amylase preparation (t.q.) | 0-3 |
| Perfume | 0.05-0.25 |
| Dye solution | 0.0-1 |
| Zinc salt | 0.1-0.3 |
| Sodium sulfate | 0.0-10 |
| Water | 0.0-1.5 |
| pH adjuster (e.g., citric acid) | 0-1.5 |
| Processing aids | 0-5 |

The activity-equivalent use of the relevant enzyme ensures that the respective enzymatic properties, for example the cleaning performance on certain stains, are compared even if the ratio of active substance to total protein (the values of the specific activity) diverges. In general, a low specific activity can be compensated by adding a larger amount of protein. Furthermore, the enzymes to be examined can also be used in the same amount of substance or amount by weight if the enzymes to be examined have a different affinity for the test substrate in an activity test. The expression “same amount of substance” in this context relates to a molar use of the enzymes to be examined. The expression “equal weight” relates to the use of the same weight of the enzymes to be examined.

Preferred embodiments of proteases according to the invention achieve such advantageous cleaning performance even at low temperatures, in particular within temperature ranges of between approximately 20° C. and approximately 60° C., preferably between approximately 20° C. and approximately 40° C. and further preferably between approximately 30° C. and approximately 20° C., and particularly preferably approximately 20° C.

Methods for determining protease activity are familiar to and routinely used by a person skilled in the art in the field of enzyme technology. For example, such methods are disclosed in Tenside [Surfactants], volume 7 (1970), pp. 125-132. Alternatively, the protease activity can be determined via the release of the chromophore para-nitroaniline (pNA) from the substrate suc-L-Ala-L-Ala-L-Pro-L-Phe-p-nitroanilide (AAPF). The protease cleaves the substrate and releases pNA. The release of the pNA causes an increase in absorbance at 410 nm, the time profile of which is a measure of the enzymatic activity (cf. Del Mar et al., 1979). The measurement is carried out at a temperature of 25° C., a pH of 8.6 and a wavelength of 410 nm. The measurement time is 5 min and the measurement interval is 20 s to 60 s. The protease activity is usually expressed in protease units (PU). Suitable protease activities are 2.25, 5 or 10 PU per ml of washing liquor, for example. However, protease activity is not equal to zero.

An alternative test for determining the proteolytic activity of the proteases according to the invention is an optical measurement method, preferably a photometric method. The test suitable for this purpose comprises protease-dependent cleavage of the substrate protein casein. The casein is cleaved by the protease into a plurality of smaller partial products. The entirety of these partial products has an increased absorption at 290 nm with respect to non-cleaved casein, wherein this increased absorption can be determined using a photometer, and thus a conclusion can be drawn regarding the enzymatic activity of the protease.

The protein concentration can be determined using known methods, e.g., the BCA method (bicinchoninic acid; 2,2′-bichinolyl-4,4′-dicarboxylic acid) or the Biuret method (Gornall et al. J. Biol. Chem. 177 (1948): 751-766). In this regard, the active protein concentration can be determined via titration of the active centers using a suitable irreversible inhibitor and determination of the residual activity (cf. Bender et al., J. Am. Chem. Soc. 88, 24 (1966): 5890-5913).

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least one amino acid substitution selected from the group consisting of N97E, R99E, R99I, R99N, R99V, R99T, D127E and M211C in at least one of the positions corresponding to positions 97, 99, 127 and 211.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least two amino acid substitutions selected from the group consisting of N97E, R99E, R99I, R99N, R99V, R99T, D127E and M211C in at least two of the positions corresponding to positions 97, 99, 127 and 211.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least three amino acid substitutions selected from the group consisting of N97E, R99E, R99I, R99N, R99V, R99T, D127E and M211C in at least three of the positions corresponding to positions 97, 99, 127 and 211.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least four amino acid substitutions selected from the group consisting of N97E, R99E, R99I, R99N, R99V, R99T, D127E and M211C in at least four of the positions corresponding to positions 97, 99, 127 and 211.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least two amino acid substitutions selected from the group consisting of N97E, R99E, R99I, R99N, R99V, R99T, D127E and M211C in at least two of the positions corresponding to positions 97, 99, 127 and 211, and wherein the amino acid substitution combination is selected from the combination consisting of N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least three amino acid substitutions selected from the group consisting of N97E, R99E, R99I, R99N, R99V, R99T, D127E and M211C in at least three of the positions corresponding to positions 97, 99, 127 and 211, and wherein the amino acid substitution combination is selected from the combination consisting of N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least four amino acid substitutions selected from the group consisting of N97E, R99E, R99I, R99N, R99V, R99T, D127E and M211C in at least four of the positions corresponding to positions 97, 99, 127 and 211, and wherein the amino acid substitution combination is selected from the combination consisting of N97E-R99E-D127E-M211C, N97E-R99I-D127E-M211C, N97E-R99N-D127E-M211C, N97E-R99V-D127E-M211C, N97E-R99T-D127E-M211C.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has an amino acid substitution or amino acid substitution combination that is selected from the combination consisting of N97E, R99E, R99I, R99N, R99V, R99T, D127E, M211C, N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, N97E-R99E-D127E-M211C, N97E-R99I-D127E-M211C, N97E-R99N-D127E-M211C, N97E-R99V-D127E-M211C and N97E-R99T-D127E-M211C, preferably N97E, R99E, R99I, R99N, R99V, R99T, D127E, M211C, D127E-M211C and N97E-R99I.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has an amino acid substitution or amino acid substitution combination that is selected from the combination consisting of N97E, R99E, R99I, R99N, R99V, R99T, D127E, M211C, D127E-M211C and N97E-R99I.

In particularly preferred embodiments, the protease according to the invention comprises an amino acid substitution combination selected from the group consisting of N97E, R99E, R99I, R99N, R99V, R99T, D127E, M211C, N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, N97E-R99E-D127E-M211C, N97E-R99I-D127E-M211C, N97E-R99N-D127E-M211C, N97E-R99V-D127E-M211C and N97E-R99T-D127E-M211C, preferably N97E, R99E, R99I, R99N, R99V, R99T, D127E, M211C, D127E-M211C and N97E-R99I, wherein the numbering is based on the numbering according to SEQ ID NO:1 in each case, and wherein the protease does not comprise any further modifications besides the mentioned amino acid substitutions.

In particularly preferred embodiments, the protease according to the invention comprises an amino acid substitution combination selected from the group consisting of N97E, R99E, R99I, R99N, R99V, R99T, D127E, M211C, D127E-M211C and N97E-R99I, wherein the numbering is based on the numbering according to SEQ ID NO:1 in each case, and wherein the protease does not comprise any further modifications besides the mentioned amino acid substitutions.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least one amino acid substitution selected from the group consisting of N97E, R99E, R99I, R99N, R99V, R99T, D127E and M211C in at least one of the positions corresponding to positions 97, 99, 127 and 211, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least two amino acid substitutions selected from the group consisting of N97E, R99E, R99I, R99N, R99V, R99T, D127E and M211C in at least two of the positions corresponding to positions 97, 99, 127 and 211, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least three amino acid substitutions selected from the group consisting of N97E, R99E, R99I, R99N, R99V, R99T, D127E and M211C in at least three of the positions corresponding to positions 97, 99, 127 and 211, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least four amino acid substitutions selected from the group consisting of N97E, R99E, R99I, R99N, R99V, R99T, D127E and M211C in at least four of the positions corresponding to positions 97, 99, 127 and 211, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least two amino acid substitutions selected from the group consisting of N97E, R99E, R99I, R99N, R99V, R99T, D127E and M211C in at least two of the positions corresponding to positions 97, 99, 127 and 211, and wherein the amino acid substitution combination is selected from the combination consisting of N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least three amino acid substitutions selected from the group consisting of N97E, R99E, R99I, R99N, R99V, R99T, D127E and M211C in at least three of the positions corresponding to positions 97, 99, 127 and 211, and wherein the amino acid substitution combination is selected from the combination consisting of N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has, based on the numbering according to SEQ ID NO:1, at least four amino acid substitutions selected from the group consisting of N97E, R99E, R99I, R99N, R99V, R99T, D127E and M211C in at least four of the positions corresponding to positions 97, 99, 127 and 211, and wherein the amino acid substitution combination is selected from the combination consisting of N97E-R99E-D127E-M211C, N97E-R99I-D127E-M211C, N97E-R99N-D127E-M211C, N97E-R99V-D127E-M211C, N97E-R99T-D127E-M211C, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has an amino acid substitution or amino acid substitution combination selected from the combination consisting of N97E, R99E, R99I, R99N, R99V, R99T, D127E, M211C, N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, N97E-R99E-D127E-M211C, N97E-R99I-D127E-M211C, N97E-R99N-D127E-M211C, N97E-R99V-D127E-M211C and N97E-R99T-D127E-M211C, preferably N97E, R99E, R99I, R99N, R99V, R99T, D127E, M211C, D127E-M211C and N97E-R99I, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In preferred embodiments, the protease according to the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease has an amino acid substitution or amino acid substitution combination selected from the combination consisting of N97E, R99E, R99I, R99N, R99V, R99T, D127E, M211C, D127E-M211C and N97E-R99I, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In particularly preferred embodiments, the protease according to the invention comprises an amino acid substitution combination selected from the group consisting of N97E, R99E, R99I, R99N, R99V, R99T, D127E, M211C, N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, N97E-R99E-D127E-M211C, N97E-R99I-D127E-M211C, N97E-R99N-D127E-M211C, N97E-R99V-D127E-M211C and N97E-R99T-D127E-M211C, preferably N97E, R99E, R99I, R99N, R99V, R99T, D127E, M211C, D127E-M211C and N97E-R99I, wherein the numbering is based on the numbering according to SEQ ID NO:1 in each case, and wherein the protease does not comprise any other modifications besides the mentioned amino acid substitutions, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

In particularly preferred embodiments, the protease according to the invention comprises an amino acid substitution combination selected from the group consisting of N97E, R99E, R99I, R99N, R99V, R99T, D127E, M211C, D127E-M211C and N97E-R99I, wherein the numbering is based on the numbering according to SEQ ID NO:1 in each case, and wherein the protease does not comprise any other modifications besides the mentioned amino acid substitutions, wherein the protease exhibits improved cleaning performance in comparison with a reference protease (in particular the wild-type protease according to SEQ ID NO:1) in at least one protease-sensitive stain preferably selected from the group consisting of stains containing blood, egg (yolk), milk, meat and other proteins, preferably containing egg (yolk) and/or milk, wherein the cleaning performance, as described in Example 2, is determined.

Proteases according to the invention have increased catalytic activity in washing and/or cleaning agents, in particular liquid textile washing agents. In various embodiments, the proteases according to the invention can exhibit a proteolytic activity that is at least 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130% or more based on the wild type (SEQ ID NO:1). Such performance-enhanced proteases allow improved cleaning results on protease-sensitive stains. Such performance-enhanced cleaning results on protease-sensitive stains can be achieved within different temperature ranges, for example within a range of from approximately 0° C. to approximately 100° C., preferably approximately 20° C. to approximately 60° C., more preferably approximately 20° C. to approximately 40° C., particularly preferably approximately 20° C. to approximately 30° C., very particularly preferably approximately 20° C.

The proteases according to the invention have greater stability in washing or cleaning agents, in particular liquid textile washing agents, for example with respect to surfactants and/or bleaching agents and/or chelators, and/or with respect to temperature effects, in particular high temperatures of, for example, between approximately 40° C. and approximately 60° C., and/or with respect to acidic or alkaline conditions and/or with respect to changes in pH and/or with respect to denaturing or oxidizing agents and/or with respect to (auto) proteolytic degradation and/or with respect to an alteration in redox conditions. Performance-enhanced and/or more stable protease variants are therefore provided by particularly preferred embodiments of the invention. Performance-enhanced and more stable protease variants are provided by further very particularly preferred embodiments of the invention. Such advantageous embodiments of proteases according to the invention consequently allow for improved cleaning results on protease-sensitive stains, which are preferably selected from the group consisting of stains containing blood, egg (yolk), milk, meat and other proteins, preferably containing egg (yolk) and/or milk.

The proteases according to the invention exhibit enzymatic activity, i.e., they are capable of hydrolyzing peptides and proteins, in particular in a washing or cleaning agent, preferably a liquid textile washing agent. A protease according to the invention is therefore an enzyme that catalyzes the hydrolysis of amide/peptide bonds in protein/peptide substrates and is thereby capable of cleaving proteins or peptides. Advantageously, the washing and cleaning agent, in particular liquid textile washing agent, according to the invention has an improved cleaning performance, in particular with respect to the removal of protease-sensitive stains preferably selected from the group consisting of stains containing blood, egg (yolk), milk, meat and other proteins, preferably containing egg (yolk) and/or milk. The washing and cleaning agent, in particular liquid textile washing agent, according to the invention is particularly preferably suitable for removing protease-sensitive stains selected from the group consisting of stains containing blood, egg (yolk), milk, meat and other proteins. Very particularly preferably, the washing and cleaning agent, in particular liquid textile washing agent, according to the invention is suitable for removing stains containing egg (yolk) and/or milk.

Furthermore, a protease according to the invention is preferably a mature protease, i.e., the catalytically active molecule without a signal peptide/signal peptides and/or a propeptide/propeptides. Unless otherwise stated, the sequences indicated also refer to mature (processed) enzymes in each case.

In various embodiments of the invention, the protease is a free enzyme. This means that the protease can act directly with all components of an agent and, if the agent is a liquid agent, the protease is directly in contact with the solvent of the agent (e.g., water). In other embodiments, an agent may contain proteases that form an interaction complex with other molecules or that contain a “coating.” In this case, one or more protease molecule(s) can be separated from the other constituents of the agent by a structure surrounding them. Such a separating structure can arise due to, but is not limited to, vesicles, such as a micelle or a liposome. However, the surrounding structure may also be a virus particle, a bacterial cell or a eukaryotic cell. In various embodiments, an agent may contain cells of, for example, Bacillus pumilus or Bacillus subtilis or other expression strains that express the protease according to the invention, or cell culture supernatants of such cells.

The identity of nucleic acid or amino acid sequences is determined by a sequence comparison. This sequence comparison is based on the BLAST algorithm established and commonly used in the prior art (cf. e.g., Altschul et al. (1990) “Basic local alignment search tool,” J. Mol. Biol., 215:403-410, and Altschul et al. (1997): “Gapped BLAST and PSI-BLAST: a new generation of protein database search programs,” Nucleic Acids Res., 25:3389-3402) and occurs in principle by similar sequences of nucleotides or amino acids in the nucleic acid or amino acid sequences being assigned to one another. A tabular assignment of the relevant positions is referred to as an alignment. A further algorithm available in the prior art is the FASTA algorithm. Sequence comparisons (alignments), in particular multiple sequence comparisons, are created using computer programs. The Clustal series (cf. e.g., Chenna et al. (2003): “Multiple sequence alignment with the Clustal series of programs,” Nucleic Acid Res. 31:3497-3500), T-Coffee (cf. e.g., Notredame et al. (2000): “T-Coffee: A novel method for multiple sequence alignments,” J. Mol. Biol., 302:205-217) or programs based on these programs or algorithms, for example, are frequently used. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 for sequence alignment at amino acid level). Unless stated otherwise, the sequence identity indicated herein is determined using the BLAST algorithm.

Such a comparison also allows a conclusion to be drawn about the similarity of the compared sequences to one another. It is usually indicated in percent identity, i.e., the proportion of identical nucleotides or amino acid functional groups in said sequences or in an alignment of corresponding positions. In the case of amino acid sequences, the broader concept of homology takes conserved amino acid exchanges into account, i.e., amino acids having similar chemical activity, because these usually perform similar chemical activities within the protein. Therefore, the similarity of the compared sequences can also be indicated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small, regions. Unless otherwise stated, however, identity or homology information in the present application relates to the entire length of the particular nucleic acid or amino acid sequence indicated.

In the context of the present invention, the indication that an amino acid position corresponds to a numerically designated position in SEQ ID NO:1 therefore means that the corresponding position is associated with the numerically designated position in SEQ ID NO:1 in an alignment as defined above. Furthermore, the assignment of the positions is based on the mature protein. This assignment is also to be used in particular when the amino acid sequence of a protease according to the invention comprises a higher or lower number of amino acid functional groups than the protease according to SEQ ID NO:1. Proceeding from the stated positions in the amino acid sequence of the protease according to SEQ ID NO:1, the alteration positions in a protease according to the invention are those which are assigned to said positions in an alignment.

In addition to the amino acid alterations explained above, proteases according to the invention can have further amino acid alterations, in particular amino acid substitutions, insertions or deletions. Such proteases are, for example, developed by targeted genetic modification, i.e., by mutagenesis methods, and optimized for specific applications or with regard to specific properties (e.g., with regard to their catalytic activity, stability, etc.). Furthermore, nucleic acids according to the invention can be introduced into recombination approaches and thus used to produce completely novel proteases or other polypeptides. The aim is to introduce targeted mutations such as substitutions, insertions or deletions into the known molecules in order, e.g., to improve the cleaning performance of enzymes according to the invention. For this purpose, in particular the surface charges and/or the isoelectric point of the molecules and thus their interactions with the substrate can be altered. For instance, the net charge of the enzymes can be altered in order to influence the substrate binding, in particular for use in washing and cleaning agents. Alternatively or additionally, the stability or catalytic activity of the protease can be increased by one or more corresponding mutations and its cleaning performance can thereby be improved. Advantageous properties of individual mutations, e.g., individual substitutions, can complement one another. A protease that has already been optimized with regard to specific properties, for example with regard to its stability on storage and/or activity and/or its tolerance in relation to the substrate spectrum, can therefore also be developed within the scope of the invention.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Several or alternative exchanges within the same polypeptide chain are separated by slashes. “130D/V” thus means that position 130 has mutated to D or V. In the case of insertions, additional amino acids are named according to the sequence position. In the case of deletions, the missing amino acid is replaced by a symbol, for example a star or a dash, or a A is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

The invention therefore also relates to a protease which is characterized in that it is obtained from a protease as described herein as the starting molecule by single or multiple conservative amino acid substitution, the protease in the numbering according to SEQ ID NO:1 having at least one of the above-described amino acid substitutions. The term “conservative amino acid substitution” means the exchange (substitution) of one amino acid functional group for another amino acid functional group, with this exchange not resulting in a change to the polarity or charge at the position of the exchanged amino acid, e.g., the exchange of a nonpolar amino acid functional group for another nonpolar amino acid functional group. Conservative amino acid substitutions within the context of the invention include, for example: G=A=S, I=V=L=M, D=E, N=Q, K=R, Y=F, S=T, G=A=I=V=L=M=Y=F=W=P=S=T.

Alternatively or additionally, the protease is characterized in that it is obtainable from a protease according to the invention as the starting molecule by fragmentation, deletion, insertion or substitution mutagenesis and comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease has, in each case based on the numbering according to SEQ ID NO:1, at least one amino acid substitution selected from the group consisting of N97E, R99E, R99I, R99N, R99V, R99T, D127E and M211C in at least one of the positions corresponding to positions 97, 99, 127 and 211.

For instance, it is possible to delete individual amino acids at the termini or in the loops of the enzyme without the proteolytic activity being lost or diminished in the process. Furthermore, such fragmentation, deletion mutagenesis, insertion mutagenesis or substitution mutagenesis can also, for example, reduce the allergenicity of the enzymes in question and thus improve their overall applicability. Advantageously, the enzymes retain their proteolytic activity even after mutagenesis, i.e., their proteolytic activity at least corresponds to that of the starting enzyme, i.e., in a preferred embodiment, the proteolytic activity amounts to at least 100%, preferably at least 105%, more preferably at least 110%, even more preferably at least 120% or more, of the activity of the starting enzyme. Further substitutions can also demonstrate advantageous effects. Both individual and multiple contiguous amino acids can be replaced with other amino acids.

Advantageous positions for sequence alterations, in particular substitutions, of the protease according to SEQ ID NO:1, which are preferably of significance when transferred to homologous positions of the proteases according to the invention and which impart advantageous functional properties to the protease, are therefore the positions which correspond to the positions described herein in an alignment, i.e., in the numbering according to SEQ ID NO:1. The following amino acid functional groups are located in the wild-type protease molecule at the mentioned positions: 97N, 99R, 127D, 211M.

Further confirmation of the correct assignment of the amino acids to be altered, i.e., in particular their functional correspondence, can be provided by comparative tests, based on which the two positions assigned to one another on the basis of an alignment in the two proteases compared with one another are altered in the same way and observation is carried out to determine whether the enzymatic activity is altered in the same way in the two proteases. If, for example, an amino acid exchange in a specific position of the protease in accordance with SEQ ID NO:1 is accompanied by an alteration of an enzymatic parameter, e.g., an increase in the KM value, and a corresponding alteration of the enzymatic parameter, e.g., likewise an increase in the Ky value, is observed in a protease variant in accordance with the invention of which the amino acid exchange has been achieved by the same introduced amino acid, this can therefore be considered to be confirmation of the correct assignment.

All of these aspects are also applicable to the methods according to the invention for producing a protease.

A method according to the invention for producing a protease involves introducing at least one amino acid substitution selected from the group consisting of N97E, R99E, R99I, R99N, R99V, R99T, D127E and M211C into a starting molecule that has an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% or 100% identical to the amino acid sequence indicated in SEQ ID NO:1 over its entire length in at least one of the positions that, based on the numbering according to SEQ ID NO:1, correspond to positions 97, 99, 127 and 211.

A method according to the invention can also comprise one or more of the following method steps:
(a) introducing a single or multiple conservative amino acid substitution, wherein the protease comprises, in each case based on the numbering according to SEQ ID NO:1, at least one amino acid substitution selected from the group consisting of N97E, R99E, R99I, R99N, R99V, R99T, D127E and M211C in at least one of the positions that correspond to positions 97, 99, 127 and 211;
(b) altering the amino acid sequence by fragmentation, deletion, insertion or substitution mutagenesis such that the protease comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease comprises, in each case based on the numbering according to SEQ ID NO:1, at least one amino acid substitution selected from the group consisting of N97E, R99E, R99I, R99N, R99V, R99T, D127E and M211C in at least one of the positions that correspond to positions 97, 99, 127 and 211.

All embodiments also apply to the methods according to the invention.

In a further embodiment of the invention, a protease as previously described is stabilized, in particular by means of one or more mutations, e.g., substitutions, or by coupling to a polymer. An increase in stability during storage and/or during use, for example during the washing process, results in the enzymatic activity lasting longer and thus improved cleaning performance. In principle, all stabilization options described and/or expedient in the prior art are conceivable. Preference is given to those stabilizations which are achieved via mutations of the enzyme itself because such stabilizations do not require any further working steps after the recovery of the enzyme. Examples of sequence alterations suitable for this purpose are specified above. Further suitable sequence alterations are known from the prior art.

Further possibilities for stabilization are, for example:
altering the binding of metal ions, in particular the calcium binding sites, for example, by exchanging one or more of the amino acid(s) involved in the calcium binding for one or more negatively charged amino acids and/or by introducing sequence alterations in at least one of the sequences of the two amino acids arginine/glycine;
protecting against the influence of denaturing agents such as surfactants by mutations which cause an alteration of the amino acid sequence on or at the surface of the protein;
exchanging amino acids that are close to the N-terminus for those that presumably come into contact with the rest of the molecule via non-covalent interactions and thus contribute to maintaining the globular structure.

Preferred embodiments are those in which the enzyme is stabilized in a plurality of ways because a plurality of stabilizing mutations act additively or synergistically.

Alternatively or additionally, the protease according to the invention can be combined with at least one reversible inhibitor/stabilizer compound selected from the group consisting of peptide inhibitors, in particular peptide aldehydes, polyols, in particular glycerol and 1,2-propylene glycol, benzamidine hydrochloride, borax, boric acids, boronic acids or the salts or esters or derivatives thereof, in particular phenylboronic acid derivatives or 4-formylphenylboronic acid (4-FPBA), antioxidants and combinations thereof in order to further increase the stability of the protease in washing and cleaning agents. Particularly preferred reversible protease inhibitors include boric acid, 4-FPBA and peptide inhibitors.

In the context of the present invention, “phenylboronic acid derivative” is understood to mean a compound of the following formula:

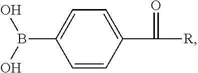

where R is hydrogen, a hydroxyl group, a C₁₋₆ alkyl group, a substituted C₁₋₆ alkyl group, a C₁₋₆ alkenyl, or a substituted C₁₋₆ alkenyl group. Preferably, the functional group R in the phenylboronic acid derivative is a C₁₋₆ alkyl group and, among these, is more preferably —CH₃, —CH₃CH₂, or —CH₃CH₂CH₂. More preferably, the functional group R in the phenylboronic acid derivative is hydrogen. The phenylboronic acid derivative 4-formylphenylboronic acid (4-FPBA) is particularly preferred.

The inhibitor/stabilizer compound used can be boric acid.

In preferred embodiments, the agent according to the invention is substantially free of boron-containing compounds. “Substantially free from boron-containing compounds” in this context means that the agents according to the invention contain less than 2 wt. %, preferably less than 1 wt. %, more preferably less than 0.5 wt. %, and particularly preferably less than 0.1 wt. %, of boron-containing compounds, based on the total weight of the agent. In particularly preferred embodiments, the agents according to the invention are free of boron-containing compounds, i.e., in particular they do not contain any boric acid and/or phenylboronic acid derivatives.

In various embodiments, the enzyme and the inhibitor/stabilizer compound may be pre-formulated in an enzyme composition. As is apparent from the previous statements, the enzyme protein forms only a fraction of the total weight of conventional enzyme preparations. Enzyme preparations that are preferably used contain between 0.1 and 40 wt. %, preferably between 0.2 and 30 wt. %, particularly preferably between 0.4 and 20 wt. %, and in particular between 0.8 and 10 wt. % of the enzyme protein. In such compositions, the inhibitor/stabilizer compound can be contained in an amount of from 0.05 to 35 wt. %, preferably from 0.05 to 10 wt. %, based on the total weight in the enzyme composition. This enzyme composition can be used in agents according to the invention, in amounts leading to the final concentrations in the agent as indicated above.

A further subject matter of the invention is a protease as described herein, characterized in that it has at least one chemical modification. A protease having such an alteration is referred to as a derivative, i.e., the protease is derivatized. In the context of the present application, derivatives are thus understood to mean proteins whose pure amino acid chain has been chemically modified. Such derivatizations can be achieved, for example, in vivo by the host cell that expresses the protein. In this regard, couplings of low-molecular-weight compounds such as lipids or oligosaccharides are particularly noteworthy. Derivatizations can also be made in vitro, for instance by means of chemical conversion of a side chain of an amino acid or by means of covalent bonding of another compound to the protein. For example, it is possible to couple amines to carboxyl groups of an enzyme in order to alter the isoelectric point. Another such compound may also be another protein that is bound to a protein according to the invention via bifunctional chemical compounds, for example. Derivatization is likewise understood to mean covalent bonding to a macromolecular carrier or a non-covalent inclusion in suitable macromolecular cage structures. Derivatizations may, for example, affect the substrate specificity or bonding strength to the substrate or cause a temporary blockage of the enzymatic activity if the coupled substance is an inhibitor. This can be expedient, for example, for the period of storage. Such modifications may further affect the stability or enzymatic activity. They can also be used to reduce the allergenicity and/or immunogenicity of the protein and for example increase its skin compatibility. For example, couplings with macromolecular compounds, for example polyethylene glycol, can improve the protein in terms of stability and/or skin compatibility. Derivatives of a protein according to the invention can also be understood in the broadest sense to be preparations of these proteins. A protein can, depending on the recovery, processing or preparation thereof, be combined with various other substances, e.g., from the culture of the producing microorganisms. A protein may also have been deliberately mixed with other substances, e.g., to increase its storage stability. Therefore, all preparations of a protein according to the invention are also in accordance with the invention. This is also independent of whether or not it actually exhibits this enzymatic activity in a particular preparation. This is because it may be desirable for it to have no activity or only a small amount of activity during storage and to only exhibit its enzymatic function at the time of use. This can be controlled via appropriate accompanying substances, for example. In particular, the joint preparation of proteases with specific inhibitors is possible in this regard. Within the scope of the present invention, of all the proteases or protease variants and/or derivatives described herein, those with a storage stability and/or catalytic activity and/or substrate tolerance and/or cleaning performance that is improved relative to the wild type or a starting variant are particularly preferred, wherein the catalytic activity and/or cleaning performance as described herein is determined.

A further subject matter of invention is a nucleic acid coding for a protease according to the invention, and a vector containing such a nucleic acid, in particular a cloning vector or an expression vector. These can be DNA or RNA molecules. They can be present as a single strand, as a single strand complementary to said single strand or as a double strand. In particular in the case of DNA molecules, the sequences of the two complementary strands must be taken into account in all three possible reading frames. Furthermore, it must be taken into account that different codons, i.e., base triplets, can code for the same amino acids such that a certain amino acid sequence can be coded by a plurality of different nucleic acids. Due to this degeneracy of the genetic code, all of the nucleic acid sequences which can code any of the proteases described above are included in this subject matter of the invention. A person skilled in the art is able to determine these nucleic acid sequences beyond a doubt because, despite the degeneracy of the genetic code, defined amino acids can be assigned to individual codons. Therefore, a person skilled in the art proceeding from said amino acid sequence can easily determine nucleic acids coding for said amino acid sequence. Furthermore, in the case of nucleic acids according to the invention, one or more codons can be replaced by synonymous codons. This aspect relates in particular to the heterologous expression of the enzymes according to the invention. Thus, each organism, for example a host cell of a production strain, has a certain codon usage. “Codon usage” is understood to mean the translation of the genetic code into amino acids by the relevant organism. Bottlenecks can occur in protein biosynthesis if the codons on the nucleic acid in the organism are faced with a comparatively small number of loaded tRNA molecules. Although coding for the same amino acid, this results in a codon being translated less efficiently in the organism than a synonymous codon coding for the same amino acid. Due to the presence of a higher number of tRNA molecules for the synonymous codon, this can be translated more efficiently in the organism.

It is possible for a person skilled in the art to use methods which are currently generally known, e.g., chemical synthesis or polymerase chain reaction (PCR), in conjunction with molecular biological and/or protein-chemical standard methods, to produce the corresponding nucleic acids and even complete genes on the basis of known DNA and/or amino acid sequences. Such methods are known, e.g., from Sambrook, J., Fritsch, E. F. and Maniatis, T. 2001. Molecular cloning: a laboratory manual, 3. Edition Cold Spring Laboratory Press.

For the purposes of the present invention, “vectors” are understood to mean elements consisting of nucleic acids that contain a nucleic acid according to the invention as the characteristic nucleic acid region. They are able to establish these as a stable genetic element in a species or cell line over several generations or cell divisions. Vectors are, in particular when used in bacteria, special plasmids, i.e., circular genetic elements. In the context of the present invention, a nucleic acid according to the invention is cloned into a vector. The vectors include, for example, those originating from bacterial plasmids, viruses or bacteriophages, or predominantly synthetic vectors or plasmids having elements of a wide variety of origins. With the other genetic elements present in each case, vectors are able to establish themselves as stable units in the particular host cells over several generations. They can be present extrachromosomally as separate units or can be integrated into a chromosome or chromosomal DNA. Expression vectors comprise nucleic acid sequences that allow them to replicate in the host cells containing them, preferably microorganisms, particularly preferably bacteria, and to express a contained nucleic acid there. The expression is influenced in particular by the promoter or promoters that regulate transcription. In principle, the expression can take place by the natural promoter originally located before the nucleic acid to be expressed, but also by a promoter of the host cell provided on the expression vector or also by a modified or completely different promoter of another organism or another host cell. In the present case, at least one promoter is provided for the expression of a nucleic acid according to the invention and used for the expression thereof. Expression vectors can also be regulatable, for example by changing the cultivation conditions or when a certain cell density of the host cells containing them is reached or by adding certain substances, in particular activators of gene expression. An example of such a substance is the galactose derivative isopropyl β-D-thiogalactopyranoside (IPTG), which is used as an activator of the bacterial lactose operon (lac operon). In contrast to expression vectors, the nucleic acid contained is not expressed in cloning vectors.

The invention further relates to a non-human host cell containing a nucleic acid according to the invention or a vector according to the invention, or containing a protease according to the invention, in particular one that secretes the protease into the medium surrounding the host cell. Preferably, a nucleic acid according to the invention or a vector according to the invention is transformed into a microorganism that then represents a host cell according to the invention. Alternatively, individual components, i.e., nucleic acid parts or fragments of a nucleic acid according to the invention, can also be introduced into a host cell in such a way that the resulting host cell contains a nucleic acid according to the invention or a vector according to the invention. This procedure is particularly suitable when the host cell already contains one or more constituents of a nucleic acid according to the invention or a vector according to the invention and the further constituents are then supplemented accordingly. Methods for transforming cells are established in the prior art and are well known to a person skilled in the art. In principle all cells, i.e., prokaryotic or eukaryotic cells, are suitable as host cells. Host cells which can be managed in a genetically advantageous manner, for example with regard to transformation with the nucleic acid or the vector and its stable establishment, are preferred, for example single-cell fungi or bacteria. Furthermore, preferred host cells are distinguished by good microbiological and biotechnological manageability. This relates, for example, to easy cultivation, high growth rates, low requirements for fermentation media and good production and secretion rates for foreign proteins. Preferred host cells according to the invention secrete the (transgenically) expressed protein into the medium surrounding the host cells. Furthermore, the proteases can be modified by the cells producing them after their production, e.g., by linking sugar molecules, formylations, aminations, etc. Such post-translational modifications can functionally influence the protease.

Further preferred embodiments are host cells that can be regulated in their activity owing to genetic regulatory elements that are provided, for example, on the vector but can also be present in these cells from the outset. Expression in said cells may be induced, for example, by controlled addition of chemical compounds used as activators, by changing the cultivation conditions or when a particular cell density is reached. This allows economic production of the proteins according to the invention. An example of such a compound is IPTG, as described above.

Prokaryotic or bacterial cells are preferred host cells. Bacteria are characterized by short generation times and low demands on cultivation conditions. This makes it possible to establish cost-effective cultivation methods or production methods. In addition, a person skilled in the art will have a wealth of experience in the case of bacteria in fermentation technology. Gram-negative or gram-positive bacteria can be suitable for a specific production for many different reasons to be determined experimentally in each individual case, such as nutrient sources, product formation rate, time needed, etc. In gram-negative bacteria, such as Escherichia coli, a plurality of proteins are secreted into the periplasmic space, i.e., into the compartment between the two membranes enclosing the cells. This can be advantageous for specific applications. Furthermore, gram-negative bacteria can also be designed such that they discharge the expressed proteins not only into the periplasmic space but into the medium surrounding the bacterium. Gram-positive bacteria such as bacilli or actinomycetes or other representatives of actinomycetes in contrast do not have an outer membrane, such that secreted proteins are immediately released into the medium surrounding the bacteria, usually the nutrient medium, from which the expressed proteins can be purified. They can be isolated directly from the medium or further processed. In addition, gram-positive bacteria are related or identical to most origin organisms for technically important enzymes and usually themselves form comparable enzymes, such that they have a similar codon usage and their protein synthesis apparatus is naturally aligned accordingly. Host cells according to the invention may be altered in terms of their requirements for culture conditions, have different or additional selection markers, or also express different or additional proteins. In particular, this may also involve those host cells which express a plurality of proteins or enzymes. The present invention can be applied in principle to all microorganisms, in particular to all fermentable microorganisms, particularly preferably to those of the genus Bacillus, and allows proteins according to the invention to be produced using such microorganisms. Such microorganisms then represent host cells for the purposes of the invention. In a further embodiment of the invention, the host cell is characterized in that it is a bacterium, preferably one selected from the group of the genera of Escherichia, Klebsiella, Bacillus, Staphylococcus, Corynebacterium, Arthrobacter, Streptomyces, Stenotrophomonas and Pseudomonas, more preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

The host cell may also be a eukaryotic cell, however, which is characterized in that it has a cell nucleus. The invention therefore further relates to a host cell that is characterized in that it has a nucleus. In contrast with prokaryotic cells, eukaryotic cells are capable of post-translationally modifying the protein formed. Examples thereof are fungi, such as actinomycetes or yeasts, such as Saccharomyces or Kluyveromyces. This can be particularly advantageous, for example, if the proteins are to undergo specific modifications in connection with their synthesis, which modifications make such systems possible. Modifications carried out by eukaryotic systems, in particular in connection with the protein synthesis, include, for example, the binding of low-molecular-weight compounds such as membrane anchors or oligosaccharides. Such oligosaccharide modifications can be desirable, for example, to reduce the allergenicity of an expressed protein. Coexpression with the enzymes naturally formed by such cells, such as cellulases, can also be advantageous. Furthermore, for example, thermophilic fungal expression systems can be particularly suitable for expression of temperature-resistant proteins or variants.

The host cells according to the invention are cultured and fermented in the usual manner, for example in discontinuous or continuous systems. In the first case, a suitable nutrient medium is inoculated with the host cells and the product is harvested from the medium after a period to be determined experimentally. Continuous fermentations are characterized by achieving a flow equilibrium in which cells partially die off over a comparatively long period but also grow back and, at the same time, the protein formed can be removed from the medium.

Host cells according to the invention are preferably used to produce proteases according to the invention. The invention therefore further relates to a method for producing a protease, comprising
a) cultivating a host cell according to the invention, and
b) isolating the protease from the culture medium or from the host cell.

This subject matter of the invention preferably comprises fermentation processes. Fermentation processes are known per se from the prior art and represent the actual large-scale production step, generally followed by a suitable purification method for the product produced, for example for the proteases according to the invention. All fermentation processes that are based on a corresponding method for producing a protease according to the invention represent embodiments of this subject matter of the invention. Fermentation processes that are characterized in that the fermentation is carried out via a feed strategy are considered in particular. In this case, the media constituents that are consumed by the continuous cultivation are added. As a result, considerable increases can be achieved both in the cell density and in the cell mass or dry mass and/or in particular in the activity of the protease of interest. Furthermore, the fermentation can also be designed such that unwanted metabolic products are filtered out or neutralized by adding buffers or suitable counterions. The prepared protease can be harvested from the fermentation medium. Such a fermentation process is preferred over isolation of the protease from the host cell, i.e., product preparation from the cell mass (dry mass), but requires the provision of suitable host cells or one or more suitable secretion markers or mechanisms and/or transport systems so that the host cells secrete the protease into the fermentation medium. Without secretion, the isolation of the protease from the host cell, i.e., purification thereof from the cell mass, can alternatively take place, for example, by means of precipitation with ammonium sulfate or ethanol or by means of chromatographic cleaning.

All of the above-mentioned aspects can be combined to form methods in order to produce the protease according to the invention.

A further subject of the invention is a washing and/or cleaning agent that is characterized in that it contains a protease according to the invention as described herein.

According to the invention, all conceivable types of washing or cleaning agents are to be understood as washing or cleaning agents, both concentrates and undiluted agents, for use on a commercial scale, in washing machines or for hand washing or cleaning. These include, for example, washing agents for textiles, carpets or natural fibers, for which the term “washing agents” is used. These also include, for example, dishwashing detergents for dishwashers (automatic dishwasher detergents) or manual dishwashing detergents or cleaners for hard surfaces such as metal, glass, porcelain, ceramics, tiles, stone, painted surfaces, plastics, wood or leather, for which the term cleaning agent is used, i.e., in addition to manual and automatic dishwashing detergents, also, for example, scouring agents, glass cleaners, WC rim blocks, etc. The washing and cleaning agents within the scope of the invention also include auxiliary washing agents that are added to the actual washing agent during manual or automatic textile washing in order to achieve a further effect. Furthermore, within the scope of the invention, washing and cleaning agents also include textile pre-treatment agents and post-treatment agents, e.g., agents with which the item of laundry is brought into contact before the actual washing, e.g., for dissolving stubborn soiling, and also agents which, in a step downstream of the actual textile washing, impart further desirable properties to the laundry item, such as a pleasant feel, crease resistance or low static charge. Inter alia, softeners are included in the latter agents. This also comprises agents for use in (semi-) automated washing or cleaning systems such as floor-mopping robots or wet vacuum cleaners.

Agents according to the invention, which may be in the form of powdered or granular solids, in compacted or re-compacted particulate form, homogeneous solutions or suspensions, may contain, in addition to a protease according to the invention, all known ingredients that are conventional in such agents, wherein preferably at least one further ingredient is present in the agent. Agents according to the invention can in particular contain surfactants, builders, polymers, glass corrosion inhibitors, corrosion inhibitors, bleaching agents such as peroxygen compounds, bleach activators or bleach catalysts. They may also contain water-miscible organic solvents, further enzymes, enzyme stabilizers, sequestering agents, electrolytes, pH regulators and/or further auxiliaries, such as optical brighteners, graying inhibitors, dye transfer inhibitors, foam regulators, as well as dyes and fragrances, and combinations thereof. Advantageous ingredients of agents according to the invention are disclosed in WO 2009/121725, starting at the penultimate paragraph of page 5 and ending after the second paragraph on page 13. Reference is expressly made to this disclosure and the disclosure therein is incorporated into the present patent application.

Agents according to the invention advantageously contain the protease according to the invention in an amount of 2 μg to 20 mg, preferably of 5 μg to 17.5 mg, particularly preferably of 20 μg to 15 mg, and very particularly preferably of 50 μg to 10 mg per g of the agent. In various embodiments, the concentration of the protease (active enzyme) described herein in the agent is >0 to 1 wt. %, preferably 0.0001 or 0.001 to 0.1 wt. %, based on the total weight of the agent or composition.

Agents according to the invention contain the protease according to the invention increasingly preferably in an amount of 1×10⁻⁸ to 5 wt. %, of 0.0001 to 1 wt. %, of 0.0005 to 0.5 wt. %, of 0.001 to 0.1 wt. %, in each case based on active protein and based on the total weight of the agent.

Further embodiments comprise all solid, powdery, liquid, gel-like or pasty dosage forms of agents according to the invention, which may optionally also consist of a plurality of phases and may be present in compressed or non-compressed form. Agents according to the invention can be in the form of a free-flowing powder, in particular having a bulk density of 300 g/l to 1200 g/l, in particular 500 g/l to 900 g/l or 600 g/l to 850 g/l. The solid administration forms of agents according to the invention further include extrudates, granules, tablets or pouches. Alternatively, agents according to the invention can also be in a liquid, gel or pasty form, e.g., in the form of a non-aqueous liquid washing agent or a non-aqueous paste or in the form of an aqueous liquid washing agent or an aqueous paste. Liquid agents are generally preferred. Furthermore, agents according to the invention can be in the form of a single-component system. Such agents consist of one phase. Alternatively, agents according to the invention can also consist of multiple phases. Such an agent is accordingly divided into a plurality of components.

In preferred embodiments, the agent according to the invention is a textile washing agent.

In preferred embodiments, the agent according to the invention is a liquid textile washing agent.

In preferred embodiments, the agent according to the invention is a pre-portioned washing agent, in particular a portioned washing agent unit comprising a washing agent preparation according to the invention and a water-soluble film which completely encloses the washing agent preparation.

The water-soluble film in which the washing agent preparation is packaged can comprise one or more structurally different water-soluble polymer(s). Suitable water-soluble polymer(s) are in particular polymers from the group of (optionally acetalized) polyvinyl alcohols (PVAL) and copolymers thereof. Suitable water-soluble films for use are marketed, inter alia, by the company MonoSol LLC, for example, under the names M8630, M8720, M8310, C8400 or M8900. For example, films with the name Solublon® PT, Solublon® GA, Solublon® KC or Solublon® KL by Aicello Chemical Europe GmbH or the VF-HP films by Kuraray are also suitable.

If the agents according to the invention are in liquid form, they preferably contain more than 40 wt. %, preferably 50 to 90 wt. %, and particularly preferably 60 to 80 wt. %, of water, based on their total weight.

Agents according to the invention can contain one or more surfactants, with anionic surfactants, non-ionic surfactants and mixtures thereof being particularly suitable, although cationic, zwitterionic and/or amphoteric surfactants can also be contained. The agents preferably contain 5 to 70 wt. % surfactant, preferably 35 to 60 wt. %, and more preferably 40 to 55 wt. % surfactant, based on the total weight of the agent. In preferred embodiments, the agents preferably contain 3 to 35 wt. %, preferably 5 to 30 wt. %, of surfactant, based on the total weight of the agent.

Suitable anionic surfactants are, in particular, soaps and those which contain sulfate or sulfonate groups, preferably having alkali ions as cations. Usable soaps are preferably the alkali salts of saturated or unsaturated C₁₂₋₁₈ fatty acids. Fatty acids of this kind can also be used in a not completely neutralized form. Suitable sulfate-type surfactants include the salts of sulfuric acid half-esters of C₁₂₋₁₈ fatty alcohols atoms and the sulfation products of the mentioned non-ionic surfactants having a low degree of ethoxylation. Surfactants of the sulfonate type that can be used include, for example, C₉₋₁₄ alkylbenzene sulfonates, alkane sulfonates obtained from C₁₂₋₁₈ alkanes, for example by sulfochlorination or sulfoxidation with subsequent hydrolysis or neutralization, C₁₂₋₁₈ olefin sulfonates resulting from the reaction of corresponding monoolefins with sulfur trioxide, mixtures of alkene and hydroxyalkane sulfonates, disulfonates, such as those obtained from C₁₂₋₁₈ monoolefins with terminal or internal double bonds by sulfonation with gaseous sulfur trioxide and subsequent alkaline or acidic hydrolysis of the sulfonation products, and α-sulfofatty acid esters (ester sulfonates) resulting from the sulfonation of fatty acid methyl or ethyl esters, e.g., a-sulfonated methyl esters of hydrogenated coconut, palm kernel or tallow fatty acids.

Preferably, the agent comprises, based on the total weight of the agent, 2 to 55 wt. %, preferably 3 to 35 wt. %, of anionic surfactant. Very particularly preferably, the agent comprises 3 to 25 wt. % alkylbenzene sulfonate. In addition, the agent can preferably also contain other anionic surfactants, in particular alkyl ether sulfates, and non-ionic surfactants, in particular fatty alcohol alkoxylates. These can then make up the remainder of the surfactants.

Suitable alkylbenzene sulfonates are preferably selected from linear or branched alkylbenzene sulfonates of the formula

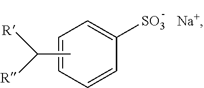

in which R′ and R″ are, independently of one another, hydrogen or alkyl, and together contain 6 to 19, preferably 7 to 15, and in particular 9 to 13, C atoms. A very particularly preferred representative is sodium dodecylbenzene sulfonate.

The alkali salts and in particular the sodium salts of the sulfuric acid half-esters of C₁₂₋₁₈ fatty alcohols, e.g. from coconut fatty alcohol, tallow fatty alcohol, lauryl alcohol, myristyl alcohol, cetyl alcohol or stearyl alcohol, or of C₁₀₋₂₀ oxo alcohols and the half-esters of secondary alcohols having these chain lengths are preferred as alk(en)yl sulfates. Alk(en)yl sulfates of the mentioned chain length that contain a synthetic straight-chain alkyl functional group prepared on a petrochemical basis and have a degradation behavior similar to that of the adequate compounds based on fat chemical raw materials are also preferred. From a washing perspective, the C₁₂₋₁₆ alkyl sulfates and C₁₂₋₁₅ alkyl sulfates and also C₁₄₋₁₅ alkyl sulfates are preferred.

The sulfuric acid monoesters of straight-chain or branched C₇₋₂₁ alcohols ethoxylated with 1 to 6 mol ethylene oxide, such as 2-methyl-branched C₉₋₁₁ alcohols with, on average, 3.5 mol ethylene oxide (EO) or C₁₂₋₁₈ fatty alcohols with 1 to 4 EO, are also suitable.

Suitable alkyl ether sulfates are, for example, compounds of the formula

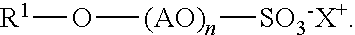

In this formula, R¹ represents a linear or branched, substituted or unsubstituted alkyl functional group, preferably a linear, unsubstituted alkyl functional group, particularly preferably a fatty alcohol functional group. Preferred functional groups R¹ are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred. Particularly preferred functional groups R¹ are derived from C₁₂₋₁₈ fatty alcohols, for example from coconut fatty alcohol, tallow fatty alcohol, lauryl, myristyl, cetyl or stearyl alcohol or from C₁₀₋₂₀ oxo alcohols. AO represents an ethylene oxide (EO) group or propylene oxide (PO) group, preferably an ethylene oxide group. The index n represents an integer from 1 to 50, preferably from 1 to 20, and in particular from 2 to 10. Very particularly preferably, n represents the numbers 2, 3, 4, 5, 6, 7 or 8. X⁺ represents a monovalent cation or the n-th part of an n-valent cation, the alkali metal ions, including Na⁺ or K⁺, being preferred in this case, with Na being most preferred. Further cations X⁺ may be selected from NH₄, ½ Zn²⁺, ½ Mg²⁺, ½ Ca²⁺, ½ Mn²⁺, and the mixtures thereof.

In various embodiments, the alkyl ether sulfate can be selected from fatty alcohol ether sulfates of the formula

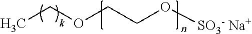

where k=11 to 19, and n=2, 3, 4, 5, 6, 7 or 8. Very particularly preferred representatives are Na—C₁₂₋₁₄ fatty alcohol ether sulfates having 2 EO (k=11-13, n=2). The degree of ethoxylation indicated represents a statistical average value which can be an integer or a fractional number for a specific product. The degrees of alkoxylation indicated represent statistical averages which can be an integer or a fractional number for a specific product. Preferred alkoxylates/ethoxylates have a narrowed homolog distribution (narrow range ethoxylates, NRE).

It has proven advantageous for cold wash performance if the agents additionally contain soap(s). Preferred agents are therefore characterized in that they contain soap(s). Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut fatty acids, palm kernel fatty acids or tallow fatty acids.

Suitable non-ionic surfactants are in particular alkyl glycosides and ethoxylation and/or propoxylation products of alkyl glycosides or linear or branched alcohols each having 8 to approximately 18 C atoms in the alkyl portion and 3 to 20, preferably 4 to 10, alkyl ether groups. Furthermore, corresponding ethoxylation and/or propoxylation products of N-alkylamines, vicinal diols, fatty acid esters, and fatty acid amides, which correspond to the above-mentioned long-chain alcohol derivatives with respect to the alkyl moiety, and of alkylphenols having 5 to 12 C atoms in the alkyl radical, may be used.

Non-ionic surfactants that are preferably used are alkoxylated, advantageously ethoxylated, in particular primary alcohols having preferably 8 to 18 C atoms and, on average, 1 to 12 mol ethylene oxide (EO) per mol of alcohol, in which the alcohol functional group can be linear or preferably methyl-branched in the 2-position, or can contain linear and methyl-branched functional groups in the mixture, as are usually present in oxo alcohol functional groups. However, alcohol ethoxylates having linear functional groups of alcohols of native origin having 12 to 18 C atoms, for example of coconut, palm, tallow fatty or oleyl alcohol, and an average of 2 to 8 EO per mol of alcohol, are particularly preferred. Preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO or 8 EO, C₁₂₋₁₈ alcohols having 3 EO, 5 EO or 7 EO, and mixtures thereof, such as mixtures of C₁₂₋₁₄ alcohol having 3 EO and C₁₂₋₁₈ alcohol having 5 EO. The degrees of ethoxylation specified represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Another class of non-ionic surfactants that are preferably used, which are used either as the sole non-ionic surfactant or in combination with other non-ionic surfactants, is alkoxylated, preferably ethoxylated or ethoxylated and propoxylated fatty acid alkyl esters, preferably having 1 to 4 carbon atoms in the alkyl chain, in particular fatty acid methyl esters.

Another class of non-ionic surfactants that can advantageously be used is the alkyl polyglycosides (APG). Usable alkyl polyglycosides satisfy the general formula

RO(G)_{z},
in which R is a linear or branched, in particular methyl-branched in the 2-position, saturated or unsaturated, aliphatic functional group having 8 to 22, preferably 12 to 18 C atoms and G is the symbol which represents a glycose unit having 5 or 6 C atoms, preferably glucose. The degree of glycosidation z is between 1 and 4, preferably between 1 and 2, and in particular between 1.1 and 1.4. Linear alkyl polyglycosides, i.e., alkyl polyglycosides in which the polyglycol functional group is a glucose functional group and the alkyl functional group is an n-alkyl functional group, are preferably used.

Non-ionic surfactants of the aminoxide type, for example N-cocoalkyl-N,N-dimethylamine oxide and N-tallow-alkyl-N,N-dihydroxyethylamine oxide, and of the fatty acid alkanolamides may also be suitable. The quantity of these non-ionic surfactants is preferably no more than that of the ethoxylated fatty alcohols, in particular no more than half thereof.

Suitable amphoteric surfactants are, for example, betaines of the formula

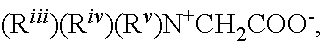

in which Rⁱⁱⁱ denotes an alkyl group, which is optionally interrupted by heteroatoms or heteroatom groups, having 8 to 25, preferably 10 to 21, carbon atoms, and R^{iv} and R^{v} denote identical or different alkyl functional groups having 1 to 3 carbon atoms, in particular C₁₀₋₁₈ alkyl dimethyl carboxymethyl betaine and C₁₁₋₁₇ alkyl amidopropyl dimethyl carboxymethyl betaine.

Suitable cationic surfactants include, inter alia, the quaternary ammonium compounds of the formula

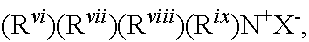

in which R^{vi} to R^{ix} represent four identical or different, in particular two long-chain and two short-chain, alkyl functional groups, and X⁻ represents an anion, in particular a halide ion, e.g., didecyldimethylammonium chloride, alkylbenzyldidecylammonium chloride, and mixtures thereof. Further suitable cationic surfactants are the quaternary surface-active compounds, in particular having a sulfonium, phosphonium, iodonium or arsonium group, which are also known as antimicrobial active ingredients. By using quaternary surface-active compounds having an antimicrobial effect, the agent can be provided with an antimicrobial effect or the antimicrobial effect that may already be present due to other ingredients can be improved.

In preferred embodiments, the washing and cleaning agent according to the invention comprises, in each case based on the total weight of the agent,
(i) 2 to 20 wt. %, preferably 3 to 17 wt. %, of anionic surfactants,
(ii) 1 to 10 wt. %, preferably 3 to 8 wt. %, of non-ionic surfactants,
(iii) 0 to 1 wt. %, preferably 0 to 0.5 wt. %, of soap, and
(iv) 0 to 5 wt. %, preferably 0 to 3 wt. %, of fatty acids.

Complexing agents are another preferred component of agents according to the invention. Particularly preferred complexing agents are the phosphonates, provided that their use is permitted by regulations. In addition to 1-hydroxyethane-1,1-diphosphonic acid, the complexing phosphonates include a number of different compounds such as diethylenetriamine penta(methylene phosphonic acid) (DTPMP). Hydroxyalkane or aminoalkane phosphonates are particularly preferred in this application. Among the hydroxyalkane phosphonates, 1-hydroxyethane-1,1-diphosphonate (HEDP) is of particular importance as a cobuilder. It is preferably used as a sodium salt, the disodium salt reacting neutrally and the tetrasodium salt reacting alkaline (pH 9). Possible amino alkane phosphonates preferably include ethylenediamine tetramethylene phosphonate (EDTMP), diethylenetriamine pentamethylene phosphonate (DTPMP) and the higher homologs thereof. They are preferably used in the form of the neutrally reacting sodium salts, e.g., as hexasodium salt of EDTMP or as the heptasodium and octasodium salt of DTPMP. Of the class of phosphonates, HEDP is preferably used as a builder. The aminoalkane phosphonates also have a pronounced heavy metal binding capacity. Accordingly, it may be preferred, in particular if the agents also contain bleach, to use aminoalkane phosphonates, in particular DTPMP, or to use mixtures of the aforementioned phosphonates. A preferred agent in the context of this application contains one or more phosphonate(s) from the group aminotrimethylene phosphonic acid (ATMP) and/or the salts thereof; ethylenediamine tetra(methylene phosphonic acid) (EDTMP) and/or the salts thereof; diethylenetriamine penta(methylene phosphonic acid) (DTPMP) and/or the salts thereof; 1-hydroxyethane-1,1-diphosphonic acid (HEDP) and/or the salts thereof; 2-phosphonobutane-1,2,4-tricarboxylic acid (PBTC) and/or the salts thereof; hexamethylenediamine tetra(methylene phosphonic acid) (HDTMP) and/or the salts thereof; nitrilotri(methylenephosphonic acid) (NTMP) and/or the salts thereof. Agents containing 1-hydroxyethane-1,1-diphosphonic acid (HEDP) or diethylenetriaminepenta(methylenephosphonic acid) (DTPMP) as phosphonates are particularly preferred. According to the invention agents may, of course, contain two or more different phosphonates.

Agents that are preferred according to the invention are characterized in that the agent contains at least one complexing agent from the group of phosphonates, preferably 1-hydroxyethane-1,1-diphosphonate, the proportion by weight of the phosphonate with respect to the total weight of the agent preferably being between 0.1 and 8.0 wt. %, more preferably 0.2 and 5.0 wt. %, even more preferably 0.3 and 3.0 wt. %, and particularly preferably 0.5-2.0 wt. %.

In further preferred embodiments, agents according to the invention are substantially free of phosphonate-containing compounds. “Substantially free of phosphonate-containing compounds” in this context means that the corresponding agents or compositions contain less than 2 wt. %, preferably less than 1 wt. %, more preferably less than 0.5 wt. %, and particularly preferably less than 0.1 wt. %, of phosphonate-containing compounds, based on the total weight of the agent. In particularly preferred embodiments, these agents/compositions are free from phosphonate-containing compounds.

Agents according to the invention further preferably contain builders, preferably at least one water-soluble and/or water-insoluble, organic and/or inorganic builder. The builders include in particular the silicates, carbonates and/organic cobuilders.

Polycarboxylates/polycarboxylic acids, polymeric polycarboxylates, aspartic acid, polyacetals, dextrins, other organic cobuilders and phosphonates are particularly noteworthy as organic cobuilders. These substance classes are described below. Organic cobuilder substances can, if desired, be contained in amounts of up to 40 wt. %, in particular up to 25 wt. %, and preferably from 1 to 8 wt. %, based on the total weight of the agent. Suitable organic builder substances are, for example, the polycarboxylic acids that can be used in the form of the free acids and/or the sodium salts thereof, where polycarboxylic acids are understood to mean the carboxylic acids which carry more than one acid function. For example, these are citric acid, adipic acid, succinic acid, glutaric acid, malic acid, tartaric acid, maleic acid, fumaric acid, saccharic acid and carboxymethyl inulines, monomeric and polymeric aminopolycarboxylic acids, in particular glycine diacetic acid, methylglycine diacetic acid, nitrilotriacetic acid (NTA), iminodisuccinates such as ethylenediamine-N,N′-disuccinic acid and hydroxyiminodisuccinates, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds such as dextrin, and polymeric (poly) carboxylic acids, polycarboxylates which can be obtained in particular by oxidizing polysaccharides or dextrins, and/or polymeric acrylic acids, methacrylic acids, maleic acids, and mixed polymers thereof, which may also contain small amounts of polymerizable substances without carboxylic acid functionality in polymerized form. If desired, such organic builder substances can be contained in amounts of up to 50 wt. %, in particular up to 25 wt. %, preferably from 10 to 20 wt. %, and particularly preferably from 1 to 5 wt. %, based on the total weight of the agent. In addition to their builder effect, the free acids typically also have the property of being an acidification component and are thus also used for setting a lower and milder pH of agents. Particularly noteworthy here are citric acid, succinic acid, glutaric acid, adipic acid, gluconic acid, and any mixtures thereof. Citric acid or salts of citric acid are particularly preferably used as builder substances. Further particularly preferred builder substances are selected from methylglycinediacetic acid (MGDA), glutamic acid diacetate (GLDA), aspartic acid diacetate (ASDA), hydroxyethyl iminodiacetate (HEIDA), iminodisuccinate (IDS), ethylenediamine disuccinate (EDDS), carboxymethyl inulin and polyaspartate.

In preferred embodiments, citric acid and/or citrate is used as the water-soluble, organic builder. Particularly preferred is the use, based on the total weight of the agent, of 0.5 to 25 wt. %, preferably 0.75 to 12.5 wt. %, more preferably 1 to 4 wt. % of citric acid and/or 0.5 to 25 wt. %, preferably 0.75 to 12.5 wt. %, more preferably 1 to 4 wt. % of citrate, preferably alkali citrate, even more preferably sodium citrate. Citric acid/citrate can each be used in the form of their hydrates, for example citric acid can be used in the form of the monohydrate, and citrate can be used in the form of the trisodium citrate dihydrate.

In more preferred embodiments, the builder substances are selected from MGDA and GLDA. As used herein, the term “MGDA” comprises, but is not limited to, methylglycine diacetic acid, α-alanine diacetic acid, N-(1-carboxyethyl)-iminodiacetic acid and N,N-bis(carboxymethyl)-DL-alanine, wherein the free acid forms and the corresponding salts, preferably alkali salts, in particular trisodium salts, are included. As used herein, the term “GLDA” includes, but is not limited to, glutamic acid diacetic acid, L-glutamic acid-N,N-diacetic acid and N,N-bis(carboxylatomethyl)-L-glutamate, wherein free acid forms and corresponding salts, preferably alkali salts, in particular tetrasodium salts, are included. Although, based on the total weight of the agent, higher MGDA or GLDA concentrations are possible, in particular up to 25 wt. %, 0.2 to 5 wt. %, preferably 0.25 to 3 wt. %, even more preferably 0.5 to 2 wt. % MGDA, preferably MGDA trisodium salt (MGDA-Na₃) is used. Even more preferable is the use, based on the total weight of the agent, of 0.2 to 5 wt. %, preferably 0.25 to 3 wt. %, even more preferably 0.5 to 2 wt. % of GLDA, preferably GLDA tetrasodium salt (GLDA-Na₄).

Polymeric polycarboxylates are also suitable as builders, e.g., the alkali metal salts of polyacrylic acid or polymethacrylic acid, e.g., those with a relative molecular mass of 500 to 70,000 g/mol. For the purpose of this application, the molar masses indicated for polymeric polycarboxylates are weight-average molar masses Mw of the respective acid form which have been determined in principle using gel permeation chromatography (GPC), a UV detector having been used. The measurement was carried out against an external polyacrylic acid standard which, due to the structural relationship to the tested polymers, yields realistic molecular weight values. These specifications differ significantly from the molecular weight specifications for which polystyrene sulfonic acids are used as the standard. The molar masses measured against polystyrene sulfonic acids are generally considerably higher than the molar masses indicated in this application. Suitable polymers are in particular polyacrylates which preferably have a molecular mass of from 2,000 to 20,000 g/mol. Due to their superior solubility, short-chain polyacrylates from this group having molar masses of from 2,000 to 10,000 g/mol, and particularly preferably of from 3,000 to 5,000 g/mol, may in turn be preferred. In addition, copolymeric polycarboxylates are suitable, in particular those of acrylic acid with methacrylic acid and those of acrylic acid or methacrylic acid with maleic acid. Copolymers of acrylic acid with maleic acid containing 50 to 90 wt. % acrylic acid and 50 to 10 wt. % maleic acid have proven to be particularly suitable. The relative molecular mass thereof, based on free acids, is generally 2,000 to 70,000 g/mol, preferably 20,000 to 50,000 g/mol, and in particular 30,000 to 40,000 g/mol.

A solid agent according to the invention preferably contains at least one water-soluble and/or water-insoluble, organic and/or inorganic builder. The water-soluble organic builder substances include the aforementioned organic builder substances.

In addition to the water-soluble organic builders mentioned above, the agents of the invention may also further contain inorganic water-soluble builders. In particular, alkali silicates, alkali carbonates, alkali hydrogen carbonates, alkali phosphates and/or sesquicarbonates, which can be present in the form of their alkaline, neutral, or acidic sodium or potassium salts, can be used as water-soluble inorganic builder materials. Small amounts of calcium carbonate may optionally also be contained in solid textile washing agents. Water-soluble crystalline and/or amorphous alkali silicates are suitable, for example. The alkali silicates that can be used in the agents according to the invention as builders preferably have a molar ratio of alkali oxide to SiO₂ of less than 0.95, in particular of from 1:1.1 to 1:12, and may be present in amorphous or crystalline form. Preferred alkali silicates are sodium silicates, in particular amorphous sodium silicates, having a molar ratio of Na₂O:SiO₂ of from 1:2 to 1:2.8. As crystalline silicates, which can be present alone or in a mixture with amorphous silicates, crystalline phyllosilicates of the general formula Na₂SiₓO₂ₓ₊₁·y H₂O are preferably used, in which x, known as the modulus, is a number from 1.9 to 22, in particular 1.9 to 4, and y is a number from 0 to 33, and preferred values for x are 2, 3 or 4. Preferred crystalline phyllosilicates are those in which x assumes the values 2 or 3 in the mentioned general formula. In particular, both β- and δ-sodium disilicates (Na₂Si₂O₅·y H₂O) are preferred. Practically water-free crystalline alkali silicates of the above general formula, in which x is a number from 1.9 to 2.1 and which are produced from amorphous alkali silicates, may also be used in agents according to the invention. In a further embodiment of agents according to the invention, a crystalline sodium phyllosilicate having a module of from 2 to 3, as can be prepared from sand and soda, is used. Crystalline sodium silicates having a module in the range of from 1.9 to 3.5 are used in a further embodiment of agents according to the invention. In agents containing amorphous and crystalline alkali silicates, the weight ratio of amorphous alkali silicate to crystalline alkali silicate is preferably from 1:2 to 2:1 and in particular from 1:1 to 2:1. Crystalline phyllosilicates of the above formula are sold by Clariant GmbH under the trade name Na-SKS, for example Na-SKS-1 (Na₂Si₂₂O₄₅·x H₂O, kenyaite), Na-SKS-2 (Na₂Si₁₄O₂₉·x H₂O, magadiite), Na-SKS-3 (Na₂Si₈O₁₇·x H₂O) or Na-SKS-4 (Na₂Si₄O₉·x H₂O, macatite). Of these, Na-SKS-5 (α-Na₂Si₂O₅), Na-SKS-7 (ß-Na₂Si₂O₅, natrosilite), Na-SKS-9 (NaHSi₂O₅·3 H₂O), Na-SKS-10 (NaHSi₂O₅·3 H₂O, kanemite), Na-SKS-11 (t-Na₂Si₂O₅) and Na-SKS-13 (NaHSi₂O₅), but in particular Na-SKS-6 (δ-Na₂Si₂O₅) are particularly suitable. In one embodiment of agents according to the invention, a granular compound made of crystalline phyllosilicate and citrate, crystalline phyllosilicate and the above-described (co) polymeric polycarboxylic acid, or alkali silicate and alkali carbonate is used, as is commercially available under the name Nabion® 15, for example. Such water-soluble inorganic builder materials are preferably contained in the agents according to the invention in amounts of 1 to 20 wt. %, in particular 5 to 15 wt. %, based on the total weight of the agent. Also of significance as water-soluble inorganic builder substances are the carbonates (and hydrogen carbonates), in particular sodium carbonate, and the phosphonic acids/phosphonates.

In preferred embodiments, agents according to the invention are substantially free of phosphate-containing compounds. “Substantially free from phosphate-containing compounds” in this context means that the corresponding agents contain less than 2 wt. %, preferably less than 1 wt. %, more preferably less than 0.5 wt. %, and particularly preferably less than 0.1 wt. %, of phosphate-containing compounds, based on the total weight of the agent. In particularly preferred embodiments, these agents are free from phosphate-containing compounds. This means that agents according to the invention do not contain any deliberately added phosphate builder.

Agents according to the invention may also contain water-insoluble builder substances. Crystalline or amorphous water-dispersible alkali aluminosilicates, in amounts of up to 50 wt. %, preferably not more than 40 wt. %, in particular from 3 to 20 wt. %, and particularly preferably from 1 to 15 wt. %, based on the total weight of the agent, are used in particular as water-insoluble inorganic builder materials. Among these, the crystalline sodium aluminosilicates in washing agent quality, in particular zeolite A, zeolite P, zeolite MAP and optionally zeolite X, either alone or in mixtures, for example in the form of a co-crystallizate of the zeolites A and X (Vegobond® AX, a commercial product from Condea Augusta S.p.A.), are preferred. Amounts close to the stated upper limit are preferably used in solid, particulate agents. Suitable aluminosilicates have, in particular, no particles having a particle size above 30 μm and preferably consist by at least 80 wt. % of particles having a size below 10 μm. The calcium binding capacity, which can be determined according to DE 2412837 A1, of said aluminosilicates is generally in the range of from 100 to 200 mg CaO per gram.

In preferred embodiments, agents according to the invention comprise a builder system comprising at least one builder, preferably in an amount of 0.5 to 50 wt. %, preferably 0.5 to 20 wt. %, particularly preferably 0.5 to 10 wt. %, based on the total weight of the agent, wherein the builder is selected from the group consisting of polycarboxylic acids such as citric acid, adipic acid, succinic acid, glutaric acid, malic acid, tartaric acid, maleic acid, fumaric acid, saccharic acids and carboxymethyl inulines or salts thereof, monomeric and polymeric aminopolycarboxylic acids such as glycine diacetic acid, methylglycine diacetic acid (MGDA), glutamic acid diacetic acid (GLDA), nitrile triacetic acid, iminodisuccinate such as ethylenediamine-N,N′-disuccinic acid and hydroxyiminodisuccinates, ethylenediaminetetraacetic acid and polyaspartic acid or salts thereof, polyphosphonic acids such as aminotris(methylenephosphonic acid), ethylenediaminetetrakis(methylenephosphonic acid), lysinetetra(methylenephosphonic acid), diethylenetriaminepenta(methylenephosphonic acid) (DTPMP) and 1-hydroxyethane-1,1-diphosphonic acid (HEDP) or salts thereof, polymeric hydroxy compounds such as dextrin, and mixtures thereof.

In preferred embodiments, agents according to the invention comprise, in each case based on the total weight of the agent,
(i) 0 to 10 wt. %, preferably 1 to 4 wt. %, of citric acid and/or citrate, preferably alkali citrate,
(ii) 0 to 40 wt. %, preferably 0 to 15 wt. %, more preferably 1 to 3 wt. %, of alkali carbonate, preferably sodium carbonate,
(iii) 0 to 20 wt. %, preferably 3 to 10 wt. %, of alkali silicate,
(iv) 0 to 10 wt. %, preferably 0.5 to 2 wt. %, of phosphonic acid and/or alkali phosphonate, particularly preferably HEDP and/or DTPMP, and/or
(v) 0 to 10 wt. %, preferably 0.5 to 3 wt. %, of aminopolycarboxylic acids, preferably MGDA and/or GLDA.

In addition to the builders described above, the agent may contain cleaning-active polymers. The proportion by weight of the cleaning-active polymers with respect to the total weight of agents according to the invention is preferably 0.1 to 20 wt. %, more preferably 1.0 to 15 wt. %, and even more preferably 2.0 to 12 wt. %.

Possible peroxygen compounds suitable for use in the agents according to the invention include, in particular, organic peroxy acids or peracid salts of organic acids, such as phthalimidopercaproic acid, perbenzoic acid, or salts of diperdodecanedioic acid, hydrogen peroxide and inorganic salts releasing hydrogen peroxide under the washing conditions, which salts include perborate, percarbonate, persilicate, and/or persulfates such as caroate, as well as hydrogen peroxide inclusion compounds such as H₂O₂-urea adducts. Hydrogen peroxide can also be produced by means of an enzymatic system, i.e., an oxidase and the substrate thereof. If solid peroxygen compounds are intended to be used, these may be used in the form of powders or granules, which may also be coated in a manner known in principle. The peroxygen compounds can be added to the washing liquor as such or in the form of the agents containing them, which in principle can contain all conventional washing, cleaning or disinfectant components. Particularly preferably, alkali percarbonate or alkali perborate monohydrate is used. If an agent according to the invention contains peroxygen compounds, they are present in amounts of preferably up to 50 wt. %, in particular from 5 to 30 wt. %, more preferably from 0.1 to 20 wt. %, based on the total weight of the agent.

In preferred embodiments, agents according to the invention are substantially free of peroxygen compounds. “Substantially free from peroxygen-containing compounds” in this context means that the corresponding agents or compositions contain less than 2 wt. %, preferably less than 1 wt. %, more preferably less than 0.5 wt. %, and particularly preferably less than 0.1 wt. %, of peroxygen-containing compounds, based on the total weight of the agent/composition. In particularly preferred embodiments, these agents/compositions are free from peroxygen-containing compounds.

Compounds which, under perhydrolysis conditions, result in aliphatic peroxocarboxylic acids having preferably 1 to 10 C atoms, in particular 2 to 4 C atoms, and/or optionally substituted perbenzoic acid, may be used in the agents as bleach activators. Suitable substances are those which carry O- and/or N-acyl groups of the stated number of C atoms and/or optionally substituted benzoyl groups. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates or carboxylates or the sulfonic or carboxylic acids thereof, in particular nonanoyloxybenzenesulfonate or isononanoyloxybenzenesulfonate or laroyloxybenzenesulfonate (NOBS or iso-NOBS or LOBS), 4-(2-decanoyloxyethoxycarbonyloxy)-benzenesulfonate (DECOBS) or decanoyloxybenzoate (DOBA), carboxylic acid anhydrides, in particular phthalic acid anhydride, acylated polyhydric alcohols, in particular triacetin, ethylene glycol diacetate, 2,5-diacetoxy-2,5-dihydrofuran and enol esters, as well as acetylated sorbitol and mannitol or the described mixtures thereof (SORMAN), acylated sugar derivatives, in particular pentaacetyl glucose (PAG), pentaacetyl fructose, tetraacetylxylose and octaacetyl lactose, acetylated, optionally N-alkylated glucamine and gluconolactone, N-acylated lactams, for example N-benzoylcaprolactam, nitriles from which perimidic acids are formed, in particular aminoacetonitrile derivatives having a quaternized nitrogen atom, and/or oxygen-transferring sulfonimines and/or acylhydrazones. The hydrophilically substituted acyl acetals and the acyl lactams are likewise preferably used. Combinations of conventional bleach activators can also be used. Such bleach activators can, in particular in the presence of the aforementioned hydrogen peroxide-yielding bleaching agents, be present in the customary quantity range, preferably in amounts of from 0.5 to 10 wt. %, and in particular 1 to 8 wt. %, based on the total weight of the agent, but are preferably entirely absent when percarboxylic acid is used as the sole bleaching agent.

In addition to or instead of the conventional bleach activators, sulfonimines and/or bleach-boosting transition metal salts or transition metal complexes may also be contained in solid agents as what are referred to as bleach catalysts.

In preferred embodiments, agents according to the invention are substantially free of bleach activators and/or are free of bleach catalysts. “Substantially free” in this context means that the corresponding agents or compositions contain, based on the total weight of the agent, less than 2 wt. %, preferably less than 1 wt. %, more preferably less than 0.5 wt. %, and particularly preferably less than 0.1 wt. %, of bleach activators and/or bleach catalysts. In particularly preferred embodiments, these agents/compositions are free of bleach activators and/or bleach catalysts.

In preferred embodiments, agents according to the invention are substantially free of bleach, i.e., substantially free of any bleaching substances, in particular free of the peroxygen compounds, bleach activators and bleach catalysts described above. “Substantially free” in this context means that the corresponding agents or compositions contain, based on the total weight of the agent, less than 2 wt. %, preferably less than 1 wt. %, more preferably less than 0.5 wt. %, and particularly preferably less than 0.1 wt. %, of bleach, i.e., any bleaching substances, in particular the peroxygen compounds, bleach activators and bleach catalysts described above. In particularly preferred embodiments, these agents/compositions are free of bleach, i.e., free of any bleaching substances, in particular free of the peroxygen compounds, bleach activators and bleach catalysts described above.

Suitable graying inhibitors or soil release active ingredients (soil release polymer) are cellulose ethers, such as carboxymethyl cellulose, methyl cellulose, hydroxyalkyl celluloses and mixed cellulose ethers, such as methyl hydroxyethyl cellulose, methylhydroxypropyl cellulose and methyl carboxymethyl cellulose. Preferably, sodium carboxymethyl cellulose, hydroxypropyl methyl cellulose and mixtures thereof and, optionally, mixtures thereof with methyl cellulose are used. The soil release active ingredients commonly used include copolyesters containing dicarboxylic acid units, alkylene glycol units and polyalkylene glycol units. The proportion of graying inhibitors and/or soil release active ingredients in agents according to the invention is generally no greater than 2 wt. % and is preferably 0.5 to 1.5 wt. %, particularly preferably 0.5 to 2 wt. %, based on the total weight of the agent.

Derivatives of diaminostilbene disulfonic acid or the alkali metal salts thereof can be contained, for example, as optical brighteners, in particular for textiles made of cellulose fibers (e.g., cotton). Salts of 4,4′-bis(2-anilino-4-morpholino-1,3,5-triazinyl-6-amino) stilbene-2,2′-disulfonic acid or compounds having a similar structure which, instead of the morpholino group, have a diethanolamino group, a methylamino group, or a 2-methoxyethylamino group are suitable, for example. Furthermore, brighteners of the substituted 4,4′-distyryl-diphenyl type can be present, e.g., 4,4′-bis-(4-chloro-3-sulfostyryl)-diphenyl. Mixtures of brighteners can also be used. Brighteners of the 1,3-diaryl-2-pyrazoline type, e.g., 1-(p-sulfoamoylphenyl)-3-(p-chlorophenyl)-2-pyrazoline, and compounds having a similarly structure are particularly suitable for polyamide fibers. The content of the agent of optical brighteners or brightener mixtures in the agent is generally no greater than 1 wt. %, preferably 0.05 to 0.5 wt. %, based on the total weight of the agent. In a preferred embodiment of the invention, the agent is free of such active ingredients.

The customary foam regulators that can be used in the agents according to the invention include, for example, polysiloxane-silicic acid mixtures, the finely divided silicic acid contained therein preferably being silanized or otherwise hydrophobized. The polysiloxanes can consist of both linear compounds and crosslinked polysiloxane resins and mixtures thereof. Further defoamers are paraffinic hydrocarbons, in particular microparaffins and paraffin waxes of which the melting point is above 40° C., saturated fatty acids or soaps having in particular 20 to 22 C atoms, for example sodium behenate, and alkali salts of phosphoric acid mono- and/or dialkyl esters, in which the alkyl chains each have 12 to 22 C atoms. Among these, sodium monoalkyl phosphate and/or dialkyl phosphate having C16-18 alkyl groups is preferably used. The proportion of foam regulators can preferably be 0.2 to 2 wt. %, particularly preferably not greater than 1 wt. %, based on the total weight of the agent.

In order to set the desired pH, agents according to the invention can contain acids that are compatible with the system and environmentally friendly, in particular citric acid, acetic acid, tartaric acid, malic acid, lactic acid, glycolic acid, succinic acid, glutaric acid, and/or adipic acid, but also mineral acids, in particular sulfuric acid or alkali hydrogen sulfates, or bases, in particular ammonium or alkali hydroxides, preferably sodium hydroxide. These types of pH regulators are contained in the agents according to the invention in amounts preferably no greater than 10 wt. %, in particular from 0.5 to 6 wt. %, particularly preferably from 0.3 to 2 wt. %, based on the total weight of the agent.

As a further component, agents according to the invention may contain an organic solvent. Adding organic solvents has an advantageous effect on the enzyme stability and cleaning performance of these agents. Preferred organic solvents are derived from the group of monohydric or polyhydric alcohols, alkanolamines or glycol ethers. The solvents are preferably selected from ethanol, n- or i-propanol, butanol, glycol, propanediol, butanediol, glycerol, diglycol, propylene diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol methyl ether, dipropylene glycol ethyl ether, methoxytriglycol, ethoxytriglycol, butoxytriglycol, 1-butoxyethoxy-2-propanol, 3-methyl-3-methoxybutanol, propylene-glycol-t-butyl ether and mixtures of these solvents. The proportion by weight of these organic solvents with respect to the total weight of the agents according to the invention is preferably 0.1 to 10 wt. %, more preferably 0.2 to 8.0 wt. %, and even more preferably 0.5 to 5.0 wt. %. A particularly preferred organic solvent which is particularly effective in stabilizing the cleaning agents is glycerol, as well as 1,2-propylene glycol. Liquid agents preferably comprise at least one polyol, preferably from the group glycerol and 1,2-propylene glycol, based on the total weight of the agent, preferably in an amount of from 0.1 to 10 wt. %, preferably 0.2 to 8.0 wt. %, and more preferably 0.5 to 5.0 wt. %. Other preferred organic solvents are the organic amines and alkanolamines. Agents according to the invention preferably contain these amines in amounts of from 0.1 to 10 wt. %, preferably from 0.2 to 8.0 wt. %, and more preferably from 0.5 to 5.0 wt. %, based on the total weight of the agent. Ethanolamine is a particularly preferred alkanolamine.

Agents according to the invention can contain only one protease according to the invention. Alternatively, they can also contain further hydrolytic enzymes or other enzymes in a concentration expedient for the effectiveness of the agent. A further embodiment of the invention is thus represented by agents that further comprise one or more further enzymes. Further enzymes which can preferably be used are all enzymes which can exhibit catalytic activity in the agent according to the invention, in particular a lipase, amylase, cellulase, hemicellulase, mannanase, tannase, xylanase, xanthanase, xytoglucanase, ß-glucosidase, pectinase, carrageenase, perhydrolase, oxidase, oxidoreductase or another protease, which is different from the proteases according to the invention, as well as mixtures thereof. Additional enzymes are contained in the agent advantageously in an amount of from 1×10⁻⁸ to 5 wt. %, based on active protein. Increasingly preferably, each further enzyme is contained in agents according to the invention in an amount of from 1×10⁻⁷ to 3 wt. %, from 0.00001 to 1 wt. %, from 0.00005 to 0.5 wt. %, from 0.0001 to 0.1 wt. % and particularly preferably from 0.0001 to 0.05 wt. %, based on active protein. Methods for determining the enzyme concentration or the active enzyme protein content are known to those skilled in the art. Particularly preferably, the enzymes exhibit synergistic cleaning performance with respect to particular dirt or stains, i.e., the enzymes contained in the agent assist one another in their cleaning performance. Very particularly preferably, such a synergism exists between the protease present according to the invention and a further enzyme of an agent according to the invention. Synergistic effects can occur not only between different enzymes but also between one or more enzymes and other ingredients of the agent according to the invention.

Automatic dishwasher detergents preferred according to the invention contain at least one protease and at least one amylase.

Textile washing agents that are preferred according to the invention, in particular liquid textile washing agents, have at least one protease and at least one amylase. In a further preferred embodiment of the invention, textile washing agents, in particular liquid textile washing agents, have at least one protease and at least one cellulase. In a further preferred embodiment, textile washing agents, in particular liquid textile washing agents, have at least one protease and at least one lipase. In a further preferred embodiment, textile washing agents, in particular liquid textile washing agents, have at least one protease, at least one amylase and at least one lipase. In a further preferred embodiment, textile washing agents, in particular liquid textile washing agents, have at least one protease, at least one amylase and at least one cellulase. In a further preferred embodiment, textile washing agents, in particular liquid textile washing agents, have at least one protease, at least one amylase, at least one cellulase and at least one lipase. Textile washing agents, in particular liquid textile washing agents, which have 3 to 10 different enzymes are particularly preferred, wherein textile washing agents which have 3 to 10 different types of enzymes may be particularly preferable in terms of the cleaning performance on a very wide range of stains.

n preferred embodiments, an agent according to the invention contains at least one enzyme and increasingly preferably at least two, three, four or five enzymes, preferably selected from the group consisting of amylases, proteases, lipases, cellulases, mannanases and mixtures thereof, in a total amount of from 0.01 to 10 wt. %, preferably from 0.1 to 8 wt. %, more preferably from 0.2 to 6 wt. %, based on active protein and the total weight of the agent.

Examples of proteases are the subtilisins BPN′ from Bacillus amyloliquefaciens and Carlsberg from Bacillus licheniformis, protease PB92, subtilisins 147 and 309, the protease from Bacillus lentus, subtilisin DY, and the enzymes thermitase, proteinase K and proteases TW3 and TW7, which in the narrower sense are associated with the subtilases but no longer with the subtilisins. Subtilisin Carlsberg is available in a developed form under the trade name Alcalase® from Novozymes. Subtilisins 147 and 309 are marketed by Novozymes under the trade names Esperase® and Savinase®, respectively. Protease variants, described, for example, in WO 95/23221, WO 92/21760 WO 2013/060621 and EP 3660151 are derived from the protease from Bacillus lentus DSM 5483. Other proteases that are suitable are, for example, the enzymes available under the trade names Durazym®, Relase®, Everlase®, Nafizym®, Natalase®, Kannase®, Progress Uno 101L® and Ovozyme® from Novozymes, the enzymes available under the trade names Purafect®, Purafect® OxP, Purafect® Prime, Excellase® and Properase®, Preferenz P100® and Preferenz P300® from Danisco/DuPont, the enzyme available under the trade name Lavergy pro 104 LS® from BASF, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease PR from Amano Pharmaceuticals Ltd., and the enzyme available under the name Proteinase K-16 from Kao Corp. The proteases from Bacillus gibsonii and Bacillus pumilus, which are disclosed in WO 2008/086916, WO 2007/131656, WO 2017/215925, WO 2021/175696 and WO 2021/175697, are particularly preferably used. Further proteases that can be used are those which are naturally present in the microorganisms Stenotrophomonas maltophilia, in particular Stenotrophomonas maltophilia K279a, Bacillus intermedius and Bacillus sphaericus.

Examples of amylases are the α-amylases from Bacillus licheniformis, Bacillus amyloliquefaciens or Bacillus stearothermophilus, as well as in particular the developments thereof that have been improved for use in washing or cleaning agents. The enzyme from Bacillus licheniformis is available from Novozymes under the name Termamyl® and from Danisco/DuPont under the name Purastar® ST. Development products of this a-amylase are available under the trade names Duramyl® and Termamyl® ultra (both from Novozymes), Purastar® OxAm (Danisco/DuPont) and Keistase® (Daiwa Seiko Inc.). The a-amylase from Bacillus amyloliquefaciens is marketed by Novozymes under the name BAN®, and derived variants from the a-amylase from Bacillus stearothermophilus are marketed under the names BSG® and Novamyl®, also by Novozymes. Others that are particularly noteworthy for this purpose are the a-amylases from Bacillus sp. A 7-7 (DSM 12368) and the cyclodextrin glucanotransferase (CGTase) from Bacillus agaradherens (DSM 9948) should be emphasized. Fusion products of all mentioned molecules can also be used. Furthermore, the developments of the α-amylase from Aspergillus niger and A. oryzae, available under the trade name Fungamyl® from Novozymes, are suitable. Other commercial products that can be advantageously used are, for example, Amylase-LT®and Stainzyme® or Stainzyme® ultra or Stainzyme® plus as well as Amplify™ 12L, Amplify Prime™ 100L or Amplify Prime™ 120L, the latter also from Novozymes, and the PREFERENZ S® series from Danisco/DuPont, comprising, for example, PREFERENZ S100®, PREFERENZ S1000® or PREFERENZ S210®. Variants of these enzymes that can be obtained by point mutations may also be used according to the invention.

Suitable cellulases include those of bacterial or fungal origin. Chemically modified or protein-engineered mutants are included. Suitable cellulases are cellulases from the genera Bacillus, Pseudomonas, Humicola, Fusarium, Thielvia, Acremonium, e.g., the fungal cellulase from Humicola insolens, Mycelophthora thermophila and Fusarium oxysporum, which are disclosed in U.S. Pat. Nos. 4,435,307, 5,648,263, 5,691,178, 5,776,757 and WO 89/09259. Particularly suitable cellulases are the alkaline or neutral cellulases with color care properties. Examples of such cellulases are cellulases which are described in EP 0495257, EP 0531372, WO 96/11262, WO 96/29397 and WO 98/08940. Examples of cellulases with endo-1,4-glucanase activity (EC 3.2.1.4) are described in WO 2002/099091, for example those having a sequence of at least 97% identity to the amino acid sequence of positions 1 to 773 of SEQ ID NO:2 of WO 2002/099091. A further example can comprise a GH44-xyloglucanase, for example a xyloglucanase enzyme having a sequence of at least 60% identity to positions 40 to 559 of SEQ ID NO:2 of WO 2001/062903. Commercially available cellulases include Celluzyme™, Carezyme™, Carezyme Premium™ Celluclean™ (e.g., Celluclean™ 5000L and Celluclean™ 4000T), Celluclean Classic™, Cellusoft™ Endolase®, Renozyme® and Whitezyme™ (Novozymes A/S), Clazinase™ and Puradax HA™ (Genencor International Inc.), KAC-500 (B)™ (Kao Corporation), Revitalenz™ 1000, Revitalenz™ 2000 and Revitalenz™ 3000 (DuPont), as well as the Ecostone® and Biotouch® series (AB Enzymes).

Suitable lipases and cutinases are those of bacterial or fungal origin. Chemically modified mutated enzymes generated by protein engineering are included. Examples include lipase from Thermomyces, e.g., from T. Lanuginosus (formerly called Humicola lanuginosa), as described in EP 0258068 and EP 0305216, Humicola, e.g., H. insolens (WO 96/13580), lipase from strains of Pseudomonas (some of these now renamed Burkholderia), e.g., P. alcaligenes or P. pseudoalcaligenes, P. cephalia, P. sp. strain SD705, P. wisconensis, GDSL-type Streptomyces lipases, cutinase from Magnaporthegrisea, cutinase from Pseudomonas mendocina, lipase from Thermobida fusca, lipase from Geobacillus stearothermophilus, lipase from Bacillus subtilis and lipase from Streptomycesgriseus and S. pristinaespiris. The lipases that can originally be obtained from Humicola lanuginosa (Thermomyces lanuginosus) or have been developed therefrom, in particular those having one or more of the following amino acid exchanges in positions D96L, T213R and/or N233R, particularly preferably T213R and N233R, proceeding from the mentioned lipase, belong to the preferred lipases. Preferred commercial lipase products include Lipolase™, Lipex™, Lipolex™ and Lipoclean™ (Novozymes A/S), Lumafast (Genencor/DuPont), Lipomax (Gist-Brocades) and Biotouch LL100 (AB Enzymes).

Suitable mannanases are, for example, Bacillus subtilis endo-β-mannanase, Bacillus sp. I633 endo-B-mannanase, Bacillus sp. AAI12 endo-β-mannanase, Bacillus sp. AA349 endo-β-mannanase, Bacillus agaradhaerens NCIMB 40482 endo-β-mannanase, Bacillus halodurans endo-B-mannanase, Bacillus clausii endo-β-mannanase, Bacillus licheniformis endo-β-mannanase, Humicola insolens endo-β-mannanase and Caldocellulosiruptor sp. endo-β-mannanase (e.g., U.S. Pat. No. 6,060,299, WO 99/64573, U.S. Pat. No. 6,566,114 and WO 99/64619).

Pectate lyases suitable for washing and cleaning agents are described, for example, in WO 2003/095638 or WO 2015/121133. Examples of suitable pectinolytic enzymes are also the enzymes and enzyme preparations available under the trade names Gamanase®, Pektinex AR®, X-Pect® or Pectaway® from Novozymes, under the trade names Rohapect UF®, Rohapect TPL®, Rohapect PTE100®, Rohapect MPER, Rohapect MA plus HC, Rohapect DA12L®, Rohapect 10L®, Rohapect BIL® from AB Enzymes, and under the trade name Pyrolase® from Diversa Corp.

In order to increase the bleaching effect, oxidoreductases, such as oxidases, oxygenases, catalases, peroxidases, such as halo, chloro, bromo, lignin, glucose, or manganese peroxidases, dioxygenases or laccases (phenoloxidases, polyphenoloxidases) can be used according to the invention. Advantageously, organic, particularly preferably aromatic compounds that interact with the enzymes are additionally added in order to potentiate the activity of the relevant oxidoreductases (enhancers) or, in the event of greatly differing redox potentials, to ensure the flow of electrons between the oxidizing enzymes and the stains (mediators).

In the cleaning agents described herein, the enzymes to be used can further be formulated together with accompanying substances, for example from fermentation. In liquid formulations, the enzymes are preferably used as liquid enzyme formulation(s).

The enzymes are generally not provided in the form of the pure protein, but rather in the form of stabilized, storable and transportable preparations. These pre-packaged preparations include, for example, the solid preparations obtained through granulation, extrusion, or lyophilization or, in particular in the case of liquid or gel agents, solutions of the enzymes, which are advantageously maximally concentrated, have a low water content, and/or are supplemented with stabilizers or other auxiliaries.

Alternatively, the enzymes can also be encapsulated, for both the solid and the liquid administration form, e.g., by spray-drying or extrusion of the enzyme solution together with a preferably natural polymer or in the form of capsules, e.g., those in which the enzymes are enclosed in a set gel, or in those of the core-shell type, in which an enzyme-containing core is coated with a water-, air-, and/or chemical-impermeable protective layer. Other active ingredients such as stabilizers, emulsifiers, pigments, bleaching agents, or dyes can additionally be applied in overlaid layers. Such capsules are applied using methods that are known per se, for example by shaking or roll granulation or in fluidized bed processes. Advantageously, such granules are low in dust, for example due to the application of polymeric film-formers, and stable in storage due to the coating.

Furthermore, it is possible to formulate two or more enzymes together such that a single granule exhibits a plurality of enzyme activities.

The enzymes can also be introduced into water-soluble films, such as those used in the formulation of washing and cleaning agents in a unit dosage form. Such a film allows the enzymes to be released after contact with water. As used herein, “water-soluble” refers to a film structure that is preferably completely water-soluble. Preferably, such a film consists of (completely or partially hydrolyzed) polyvinyl alcohol (PVA).

Agents according to the invention may comprise one or more reversible enzyme inhibitor(s)/stabilizer(s). Agents according to the invention may contain the reversible enzyme inhibitor(s)/stabilizer(s) in a concentration of 0.1 to 2 wt. %, preferably 0.3 to 1.5 wt. %, based on the total weight of the agent. If several inhibitors/stabilizers are contained, this information refers to the total concentration. These may in particular be selected from the group consisting of polyols, such as glycerol or 1,2-ethylene glycol, benzamidine hydrochloride, borax, boric acids, boronic acids or their salts or esters or derivatives, in particular phenylboronic acid derivatives or 4-formylphenylboronic acid (4-FPBA), antioxidants, special peptide compounds and combinations thereof.

In preferred embodiments, the agent according to the invention is substantially free of boron-containing compounds. “Substantially free from boron-containing compounds” in this context means that the agents according to the invention contain less than 2 wt. %, preferably less than 1 wt. %, more preferably less than 0.5 wt. %, and particularly preferably less than 0.1 wt. %, of boron-containing compounds, based on the total weight of the agent. In particularly preferred embodiments, the agents according to the invention are free of boron-containing compounds, i.e., in particular they do not contain any boric acid and/or phenylboronic acid derivatives.

A further object of the invention is a method for cleaning textiles and/or hard surfaces, in particular dishes, which is characterized in that in at least one method step, an agent according to the invention is used. In various embodiments, the described method is characterized in that the protease is used at a temperature of from approximately 0° C. to approximately 100° C., preferably approximately 20° C. to approximately 60° C., more preferably approximately 20° C. to approximately 40° C., and particularly preferably approximately 20° C.

This includes both manual and machine methods, with machine methods being preferred because they can be controlled more precisely, for example with regard to the quantities used and contact times. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent.

Because proteases according to the invention by nature already have hydrolytic activity and also exhibit said activity in media that otherwise have no cleaning power, such as in a simple buffer, an individual and/or the single step of such a method can consist of bringing a protease according to the invention into contact with the stain as the only cleaning-active component, preferably in a buffer solution or in water. This represents a further embodiment of this subject matter of the invention.

Alternative embodiments of this subject matter of the invention also include methods for treating textile raw materials or for textile care, in which a protease according to the invention is active in at least one method step. Among these, methods for textile raw materials, fibers or textiles comprising natural constituents are preferred, and very particularly for those comprising wool or silk.

The invention further relates to a method for removing protease-sensitive stains, in particular stains containing egg (yolk), milk, meat, blood and other proteins, preferably stains containing egg (yolk) and/or milk, from textiles and/or hard surfaces, in particular dishes, wherein, in at least one method step, an agent containing a protease according to the invention, as described herein, and/or a protease according to the invention, as described herein, is used.

The invention further relates to a method for improving the cleaning performance of a washing and cleaning agent, in particular a liquid textile washing agent, on at least one protease-sensitive stain, in particular selected from stains containing egg (yolk), milk, meat, blood and other proteins, preferably stains containing egg (yolk) and/or milk, wherein the agent contains a protease according to the invention, as described herein.

Finally, the invention also covers the use of proteases according to the invention, as described herein, in washing or cleaning agents, in particular liquid textile washing agents, for the (improved) removal of protease-sensitive stains, preferably selected from the group consisting of stains containing blood, egg (yolk), milk and other proteins, preferably containing egg (yolk) and/or milk, from textiles and/or hard surfaces, in particular dishes.

In a further preferred embodiment, the invention relates to the use of a protease according to the invention as described herein in a washing or cleaning agent, in particular a liquid textile washing agent, for improving the cleaning performance of such a protease-containing washing or cleaning agent, in particular liquid textile washing agent, on at least one protease-sensitive stain preferably selected from the group consisting of stains containing blood, egg (yolk), milk, meat and other proteins, preferably containing egg (yolk) and/or milk, wherein the improvement in the cleaning performance of an agent comprising the protease according to the invention compared with an agent containing a reference protease (in particular the wild-type protease according to SEQ ID NO:1), as described in Example 2, is determined, in particular within a temperature range from approximately 20° C. to approximately 40° C., preferably at 20° C.

In a further preferred embodiment, the invention relates to the use of a protease according to the invention as described herein in a washing or cleaning agent, in particular a liquid textile washing agent, for improving the cleaning performance of such a protease-containing washing or cleaning agent, in particular liquid textile washing agent, on at least one protease-sensitive stain preferably selected from the group consisting of stains containing blood, egg (yolk), milk, meat and other proteins, preferably containing egg (yolk) and/or milk, wherein the improvement in the cleaning performance of an agent containing the protease according to the invention in comparison with an agent containing a reference protease (in particular the wild-type protease according to SEQ ID NO:1), as described in Example 2, is determined, in particular within a temperature range from approximately 20° C. to approximately 40° C., preferably at 20° C., wherein the protease is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease comprises, based on the numbering according to SEQ ID NO:1, at least one amino acid substitution selected from the group consisting of N97E, R99E, R99I, R99N, R99V, R99T, D127E and M211C in at least one of the positions corresponding to positions 97, 99, 127 and 211.

All aspects, subjects, and embodiments described for the protease according to the invention and agents containing them are also applicable to these subjects of the invention. Therefore, reference is expressly made at this point to the disclosure at the corresponding point where it is indicated that this disclosure also applies to the methods and uses according to the invention.

### EXAMPLES

TABLE 1
CLEANING AGENT MATRIX USED

| | % active | Wt. % of active |
|---|---|---|
| | substance in the | substance in |
| Chemical name | raw material | formulation |
| Water | 100% | to make up to 100% |
| Citric acid | 100% | 3-5% |
| FAEOS | 70% | 5-8% |
| C12-18 fatty alcohol | 100% | 8-11% |
| ethoxylate, 7 EO | | |
| Alkyl benzene sulfonic acid | 96% | 10-14% |
| C₁₂₋₁₄ fatty acid | 30% | 2-4% |
| Monoethanolamine | 100% | 6-8% |
| NaOH | 50% | 2% |
| Glycerol | 99.5% | 3% |
| 1,2-propanediol | 100% | 8% |
| HEDP | 60% | 0.5-2% |
| Other (antifoam, SRP, enzymes, | t.q | minors |
| fragrances, DTI) | | |
| pH 8.2-8.4 | | |

TABLE 2
PROTEASES USED

| | Amino acid substitution(s) with |
|---|---|
| | respect to SEQ ID NO: 1 |
| Protease 1 (P1) | SEQ ID NO:1 (wild type) |
| Protease 2 (P2) | D127E |
| Protease 3 (P3) | M211C |
| Protease 4 (P4) | R99E |
| Protease 5 (P5) | R99I |
| Protease 6 (P6) | N97E |
| Protease 7 (P7) | R99N |
| Protease 8 (P8) | R99V |
| Protease 9 (P9) | R99T |
| Protease 10 (P10) | D127E-M211C |
| Protease 11 (P11) | N97E-R99I |

### Example 1: Determining the Protease Activity

The activity of the protease is determined by releasing the chromophore para-nitroaniline from the substrate succinyl alanine-alanine-proline-phenylalanine-para-nitroanilide (AAPF-pNA; Bachem L-1400). The release of the pNA causes an increase in extinction at 410 nm, the time profile of which is a measure of the enzymatic activity.

The measurement was carried out at a temperature of 25° C., a pH of 8.6 and a wavelength of 410 nm. The measurement time was 5 min at a measuring interval of 20 to 60 seconds.

### Measurement Approach:

10 μL AAPF solution (70 mg/mL)
1000 μL Tris/HCl (0.1 M, pH 8.6 with 0.1% Brij 35)
10 μL diluted protease solution
Kinetics produced over 5 min at 25° C. (410 nm)

### Example 2: Determining the Cleaning Performance

### Mini-Wash Test

The cleaning performance was determined in mini-wash tests with Bacillus subtilis culture supernatants containing the expressed protease variants. The supernatants were used with the same activity as the benchmark (=wild type according to SEQ ID NO:1) with a commercially available concentration for proteases in a washing agent formulation A (according to Table 1) (28.6 mg/job active enzyme protein in the washing liquor).
Conditions: 20° C. or 40° C., 16° dH water, 1 h washing time
Dosage in washing liquor: 3.17 g/L
Stains:
1. CS38 (egg yolk/pigment (dried))
2. 10N (whole egg/soot)
3. C05 (blood/milk/India ink)
4. H-MR-B (milk)
5. PC10 (milk/oil)

Die-cut fabrics with wash-relevant stains (diameter=10 mm) were placed in microtiter plates, the washing liquor containing an agent according to Table 1 was adjusted to pH=8.2 and pre-heated to 20° C. and 40° C., final concentration 3.17 g/L, liquor and enzyme were added to the stains, incubated for 1 h at 20° C. and 40° C. and 600 rpm, the liquor was then removed and the stain was rinsed several times with clear water and allowed to dry, and the brightness was determined using a colorimeter (MACH 5, Multi area color-measurement, CFTBV.nl). The lighter the fabric, the better the cleaning performance. The Y value=brightness is measured here, the higher the brighter. All measured Y values were corrected by the performance of the liquor alone (without protease) (Y_{Variant}−Y_{Blank}=ΔY_{Variant}). For each stain, AY variant was determined for each protease, and all ΔYᵥₐᵣᵢₐₙₜ values for the five stains per variant were added together to determine ΣYᵥₐᵣᵢₐₙₜ. In order to compare the cleaning performance of the variants according to the invention with the benchmark (=protease P1), ΔY_{P1} or ΣY_{P1} (for each stain) was normalized to 100%, and the relative cleaning performance of the variants according to the invention was calculated. A ≥10% increase in cleaning performance is regarded as a significant improvement in performance.

The results are summarized in Table 3.

TABLE 3
CLEANING PERFORMANCE AT 20° C. AND 40° C.

| | ΔYᵥₐᵣᵢₐₙₜ | ΔYᵥₐᵣᵢₐₙₜ | ΔYᵥₐᵣᵢₐₙₜ | ΔYᵥₐᵣᵢₐₙₜ |
|---|---|---|---|---|
| | (total) | (%) | (total) | (%) |
| | 20° C. | 20° C. | 40° C. | 40° C. |
| P1 | 48 | 100% | 77 | 100% |
| P2 | 56 | 116.5% | 74 | 95.4% |
| P3 | 54 | 112.3% | 83 | 107.0% |
| P4 | 65 | 135.2% | 92 | 118.7% |
| P5 | 56 | 116.5% | 81 | 104.4% |
| P6 | 60 | 124.8% | 87 | 112.2% |
| P7 | 58 | 120.6% | 79 | 101.9% |
| P8 | 60 | 124.8% | 85 | 109.6% |
| P9 | 56 | 116.5% | 82 | 105.8% |
| P10 | 55 | 114.4% | 76 | 98.0% |
| P11 | 64 | 133.1% | 91 | 117.4% |

The proteases according to the invention (P2 to P11) show comparable or significantly improved cleaning performance on various protease-sensitive stains in comparison to the reference protease (wild-type protease according to SEQ ID NO:1). The improved cleaning performance is particularly evident at a comparatively low temperature of 20° C. As a result, proteases according to the invention can be used not only for washing processes at commonly used temperatures of approximately 40° C., but also at low temperatures, i.e. delicate wash cycles at 20° C. (or room temperature), for example, and show significantly improved washing performance at low temperatures compared with the reference protease.

### Example 3: Example Formulations

The proteases according to the invention can be used and realize their effect in various washing and cleaning agent compositions.

TABLE 4
LIQUID WASHING AGENTS

| | Wt. % of active substance in the formulation | | | | | |
|---|---|---|---|---|---|---|
| Chemical name | A | B | C | D | E | F |
| Demineralized water | Remainder | Remainder | Remainder | Remainder | Remainder | Remainder |
| LAS | 5.5 | 20 | 15.0 | 5.5 | 21.7 | 23.5 |
| FAEOS | 7.0 | | 5.0 | | | |
| Palm kernel oleic acid | 3.0 | 8.0 | | | 7.0 | 7.4 |
| FAEO | 5.5 | | 8.0 | | | |
| C_{13/15} OXO alcohol, 8EO | | 25 | | | | |
| C₁₂₋₁₈ fatty alcohol | | | | | 22.4 | 23.4 |
| ethoxylate, 7EO | | | | | | |
| Alkyl polyglycoside | | | 4.0 | | | |
| Non-ionic surfactants | | | | 3.1 | | |
| Soap | | | 1.0 | 0.5 | | |
| HEDP | 0.5 | | | | | |
| DTPMPA 7Na | | 1.0 | 1.0 | 0.2 | 0.5 | 1.7 |
| Citric acid | 2.5 | | 3.0 | 0.23 | | |
| NaOH | 3.0 | | | 0.7 | | |
| Glycerol | 3.0 | 5.0 | | 0.5 | 9.4 | 10.2 |
| Ethanol | 1.5 | 3.0 | | | | 3.2 |
| 1,2-propanediol | | 10.0 | 12.0 | | 5.0 | 5.6 |
| Monoethanolamine | | 6.0 | 7.0 | | 6.0 | 6.1 |
| Boric acid | 1.0 | | 1.0 | 0.5 | | |
| Polyalkoxylated | | | | | 4.5 | |
| alkanolamine | | | | | | |
| Ethoxylated | | | | | 4.5 | 3.0 |
| polyethyleneimine | | | | | | |
| Protease | 6 HPE/ml | 6 HPE/ml | 6 HPE/ml | 6 HPE/ml | 6 HPE/ml | 6 HPE/ml |
| Fragrance(s) | 0.5 | 0.5 | 0.4 | 0.3 | 0.4 | 0.25 |
| DTI, SRP, other | minors | minors | minors | minors | minors | minors |
| enzymes, defoamers, etc. | | | | | | |

TABLE 5
SOLID WASHING AGENTS

| | Wt. % of active substance | | |
|---|---|---|---|
| | in the formulation | | |
| Chemical name | A | B | C |
| LAS | 12.2 | 12.0 | 10.1 |
| Sodium fatty alcohol | 4.2 | | |
| sulphate, C₁₂₋₁₈ | | | |
| Fatty alcohol, | 4.1 | 2.3 | 1.5 |
| C₁₂₋₁₈, 7 EO | | | |
| Soaps | 0.4 | | |
| Citrate | 2.0 | | |
| Sodium carbonate | 2.4 | 17.9 | 25.1 |
| Builders | 23.0 | 7.0 | 7.6 |
| Phosphonate | 1.2 | 1.1 | 1.2 |
| Polyacrylate | 0.12 | 2.8 | 3.0 |
| Carboxymethylcellulose | 2.3 | 2.0 | 1.1 |
| 2Na2 carbonate 3 H₂O₂ | 18.5 | 15.8 | |
| TAED | 10.9 | 3.5 | |
| Fragrance(s) | 0.5 | 0.3 | 0.4 |
| Protease | 6 HPE/ml | 6 HPE/ml | 6 HPE/ml |
| Sodium sulfate, foam inhibitor, | Remainder | Remainder | Remainder |
| optical brighteners, | | | |
| fragrances, other enzymes | | | |

TABLE 6
TWO-PHASE AUTOMATIC DISHWASHER DETERGENT

| Powder phase (phase A) | A1 | A2 |
|---|---|---|
| Active substance content in wt. % | | |
| (unless otherwise stated), based on | | |
| the total weight of the powder phase | | |
| Sodium percarbonate | 13.0 | 15.0 |
| Non-ionic surfactants | 4.0 | 4.0 |
| Sulfonic acid group- | 4.0 | 4.0 |
| containing polymer | | |
| HEDP (sodium salt) | 6.0 | 6.0 |
| Sodium carbonate (incl. sodium | 24.0 | 28.0 |
| hydrogen carbonate) | | |
| MGDA (trisodium salt) | 0 | 0 |
| Phyllosilicate (SKS 6 powder) | 4.0 | 4.0 |
| Sodium citrate (calculated as | 21.0 | 21.0 |
| anhydrous sodium citrate) | | |
| Amylase (Stainzyme ® Plus 24 Evity | 1.5 | 1.5 |
| T; indication as wt. % based on the | | |
| amount of preparation used, t.q.) | | |
| Protease (total active protein) | 40 mg/job | 40 mg/job |
| Misc (including perfume, dyes, | Add 100 | Add 100 |
| preservatives, fillers e.g., sodium | | |
| sulfate, bleach catalyst (MnTACN), | | |
| bleach activator (TAED), zinc | | |
| acetate, silver protection, | | |
| other enzymes) | | |

| Gel phase (phase B) | E1 | B2 |
|---|---|---|
| Active substance content in wt. % | | |
| (unless otherwise stated), based on | | |
| the total weight of the gel phase | | |
| Polymer comprising acrylic acid- | 10.0 | 11.0 |
| containing and amidopropyl | | |
| sulfonic acid-containing monomers | | |
| Glycerol | 27.0 | 25.0 |
| 1,3-propanediol | 30.0 | 30.0 |
| PEG 400 | 15.0 | 17.0 |
| PVOH | 15.0 | 14.0 |
| Misc (inter alia, process aids, pH | Add 100 | Add 100 |
| adjusters, perfume, dye) | | |
| Gelling time/min | less than 1 | less than 1 |

The phases A1 or A2 and the phases B1 or B2 can be combined with one another as desired. Total weight of both phases in a single portion of 18.5 g.

1. A synthetic protease, comprising:
an active protease with an amino acid sequence that is at least 70% identical to the amino acid sequence indicated in SEQ ID NO:1 over its entire length;
wherein, on the basis of the numbering according to SEQ ID NO:1, the protease has at least one amino acid substitution selected from the group consisting of: N97E, R99E, R99I, R99N, R99V, R99T, D127E, and M211C.

2. The protease according to claim 1, wherein the protease exhibits proteolytic activity and comprises an amino acid sequence which is at least 70% identical to the amino acid sequence indicated in SEQ ID NO:1 over its entire length, wherein the protease has an amino acid substitution or amino acid substitution combination selected from the combinations consisting of: N97E, R99E, R99I, R99N, R99V, R99T, D127E, M211C, N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, N97E-R99E-D127E-M211C, N97E-R99I-D127E-M211C, N97E-R99N-D127E-M211C, N97E-R99V-D127E-M211C and N97E-R99T-D127E-M211C, preferably N97E, R99E, R99I, R99N, R99V, R99T, D127E, M211C, D127E-M211C, and N97E-R99I.

3. The protease according to claim 1, wherein the protease comprises an amino acid or amino acid substitution combination selected from the group consisting of: N97E, R99E, R99I, R99N, R99V, R99T, D127E, M211C, N97E-R99E, N97E-R99I, N97E-R99N, N97E-R99V, N97E-R99T, N97E-D127E, N97E-M211C, R99E-D127E, R99E-M211C, R99I-D127E, R99I-M211C, R99N-D127E, R99N-M211C, R99V-D127E, R99V-M211C, R99T-D127E, R99T-M211C, D127E-M211C, N97E-R99E-D127E, N97E-R99I-D127E, N97E-R99N-D127E, N97E-R99V-D127E, N97E-R99T-D127E, N97E-M211C-D127E, N97E-R99E-M211C, N97E-R99I-M211C, N97E-R99N-M211C, N97E-R99V-M211C, N97E-R99T-M211C, N97E-D127E-M211C, R99E-D127E-M211C, R99I-D127E-M211C, R99N-D127E-M211C, R99V-D127E-M211C, R99T-D127E-M211C, N97E-R99E-D127E-M211C, N97E-R99I-D127E-M211C, N97E-R99N-D127E-M211C, N97E-R99V-D127E-M211C and N97E-R99T-D127E-M211C, preferably N97E, R99E, R99I, R99N, R99V, R99T, D127E, M211C, D127E-M211C and N97E-R99I, wherein the numbering is based on the numbering according to SEQ ID NO:1 in each case, and wherein the protease does not comprise any further modifications besides the mentioned amino acid substitutions.

4. A protease, obtained by:
(a) altering the protease according to claim 1 as the starting molecule by single or multiple conservative amino acid substitution, the protease having, on the basis of the numbering according to SEQ ID NO:1, at least one amino acid substitution selected from the group consisting of: N97E, R99E, R99I, R99N, R99V, R99T, D127E, and M211C;
(b) altering the protease according to claim 1 as the starting molecule by fragmentation, deletion, insertion or substitution mutagenesis the protease having an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, the protease having, on the basis of the numbering according to SEQ ID NO:1, and having at least one amino acid substitution selected from the group consisting of: N97E, R99E, R99I, R99N, R99V, R99T, D127E and M211C.

5. A method for producing a protease, comprising:
introducing at least one amino acid substitution selected from the group consisting of: N97E, R99E, R99I, R99N, R99V, R99T, D127E and M211C into a starting molecule that has an amino acid sequence that is identical to at least 71% of the amino acid sequence indicated in SEQ ID NO:1 over its entire length in at least one of the positions that, based on the numbering according to SEQ ID NO:1, correspond to positions 97, 99, 127 and 211.

6. The method according to claim 5, further comprising one or more of the following method steps:
(a) introducing a single or multiple conservative amino acid substitution, wherein the protease comprises, based on the numbering according to SEQ ID NO:1, at least one amino acid substitution selected from the group consisting of: N97E, R99E, R99I, R99N, R99V, R99T, D127E, and M211C;
(b) altering the amino acid sequence by fragmentation, deletion, insertion or substitution mutagenesis such that the protease comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease comprises, based on the numbering according to SEQ ID NO:1, at least one amino acid substitution selected from the group consisting of: N97E, R99E, R99I, R99N, R99V, R99T, D127E and M211C.

7. A nucleic acid coding for a protease according to claim 1.

8. A vector containing a nucleic acid according to claim 7.

9. A non-human host cell that contains a nucleic acid according to claim 7.

10. A method for producing a protease, comprising
a) cultivating a host cell according to claim 9, and
b) isolating the protease from the culture medium or from the host cell.

11. A washing or cleaning agent, containing at least one protease according to claim 1.

12. A method for cleaning textiles and/or hard surfaces, comprising: using a washing or cleaning agent according to claim 11 in at least one method step.

13. A method for removing protease-sensitive stains, comprising: using an agent according to claim 11 in at least one method step.

14. The method according to claim 12, wherein the method is carried out at a temperature of between approximately 20° C. and approximately 40° C.

15. The method of claim 12 wherein the method is performed at a temperature of between 20° C. and 40° C.

16. A nucleic acid encoding a protease obtained by a method according to claim 5.

17. A non-human host cell that contains a protease according to claim 1.
